SEQUENCE LISTING (40 sequences)

SEQ ID NO: 1 - protein, 419 aa, Methanosarcina barkeri
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 2 - DNA, 52 bp, Artificial Sequence
  primer
gcgcaggtctcagaacgtnnkggcattaacaacgacaccgaactgagcaaac

SEQ ID NO: 3 - DNA, 46 bp, Artificial Sequence
  primer
gcgcagagtaggtctcagttccacatattccgccggaatcagaatc

SEQ ID NO: 4 - DNA, 69 bp, Artificial Sequence
  primer
gcgcaggtctcaatgctgnnkccgaccctgnnkaactatnnkcgtaaactggatcgtattctgccgggc

SEQ ID NO: 5 - DNA, 45 bp, Artificial Sequence
  primer
gcgcagagtaggtctcagcatcggacgcaggcacaggtttttatc

SEQ ID NO: 6 - DNA, 56 bp, Artificial Sequence
  primer
gcgcaggaaaggtctcaaactttnnkcaaatgggcagcggctgcacccgtgaaaac

SEQ ID NO: 7 - DNA, 52 bp, Artificial Sequence
  primer
gcgcagagtaggtctcaagttaaccatggtgaattcttccaggtgttctttg

SEQ ID NO: 8 - DNA, 69 bp, Artificial Sequence
  partial sequence
ggaaacctgatcatgtagatcgaatggactctaaatccgttcagccgggttagattcccggggtttccg

SEQ ID NO: 9 - DNA, 18 bp, Artificial Sequence
  5′-leader for PyltRNACUA DNA sequence
agatcttctagactcgaa

SEQ ID NO: 10 - DNA, 16 bp, Artificial Sequence
  3′-trailer for PyltRNACUA DNA sequence
gacaagtgcggttttt

SEQ ID NO: 11 - DNA, 64 bp, Artificial Sequence
  primer
gctcaagcttcaccatggcactagcaattagccatggtgagcaagggcgaggagctgttcaccg

SEQ ID NO: 12 - DNA, 71 bp, Artificial Sequence
  primer
tccggtggatccttatcattaagcgtaatctggaacatcgtatgggtacatcttgtacagctcgtccatgc

SEQ ID NO: 13 - DNA, 87 bp, Artificial Sequence
  primer
gaaggtacccgatcgccgcggaccggtttaattaagcggccgctagttattaatagtaatcaattacggggtcattagttcatagcc

SEQ ID NO: 14 - DNA, 98 bp, Artificial Sequence
  primer
atagcggccgcttaattaaaccggtccgcggcgatcgggtaccatgcatggcggtaatacggttatccacagaatcaggggataacgcaggaaagaac

SEQ ID NO: 15 - DNA, 52 bp, Artificial Sequence
  primer
gaaggtacccgatcgactagttattaatagtaatcaattacggggtcattag

SEQ ID NO: 16 - DNA, 46 bp, Artificial Sequence
  primer
gaaaccggtccgcggaagccatagagcccaccgcatccccagcatg

SEQ ID NO: 17 - DNA, 37 bp, Artificial Sequence
  primer
taaacttaagcttgccaccatggactacaaggacgac

SEQ ID NO: 18 - DNA, 79 bp, Artificial Sequence
  primer
ggcacaggttcttgtccacccggaaaatctgcttggacagctcggtgtcgttgttgatgccgaaccgctccacgtactc

SEQ ID NO: 19 - DNA, 76 bp, Artificial Sequence
  primer
ggtggacaagaacctgtgcctgcggcctatgctgagccccaccctgtgcaactacatgcggaaactggacagaatc

SEQ ID NO: 20 - DNA, 41 bp, Artificial Sequence
  primer
atctgcagaattccaccacactggactagtggatccttatc

SEQ ID NO: 21 - DNA, 53 bp, Artificial Sequence
  primer
atgctaggatccttaattaaactagtctagttattaatagtaatcaattacgg

SEQ ID NO: 22 - DNA, 32 bp, Artificial Sequence
  primer
atgctaagatctgtcccgttgattttggtgcc

SEQ ID NO: 23 - DNA, 39 bp, Artificial Sequence
  primer
atgctaggatccagatcttctagactcgaaggaaacctg

SEQ ID NO: 24 - DNA, 54 bp, Artificial Sequence
  primer
atgctacaattgccgcgggaattcgctagcaaaaaccgcacttgtccggaaacc

SEQ ID NO: 25 - DNA, 56 bp, Artificial Sequence
  primer
cagatccgctagcaccggtgcgatcgcaccatggaggagccgcagtcagatcctag

SEQ ID NO: 26 - DNA, 49 bp, Artificial Sequence
  primer
gctcgagatctgagtccggatggcgcgccgtctgagtcaggcccttctg

SEQ ID NO: 27 - DNA, 35 bp, Artificial Sequence
  primer
ggcgcgccatccggactcagatctcgagctcaagc

SEQ ID NO: 28 - DNA, 29 bp, Artificial Sequence
  primer
taaacaagttaacaacaacaattgcattc

SEQ ID NO: 29 - DNA, 48 bp, Artificial Sequence
  primer
gttgttgggcagtgctcgggcagtgctccctgggggcagctcgtggtg

SEQ ID NO: 30 - DNA, 23 bp, Artificial Sequence
  primer
cgagcactgcccaacaacaccag

SEQ ID NO: 31 - DNA, 34 bp, Artificial Sequence
  primer
gttgttgggcagtgctcgctaagtgctccctggg

SEQ ID NO: 32 - DNA, 53 bp, Artificial Sequence
  primer
agatccgctagcaccggtgcgatcgcaccatggctagcatgactggtggacag

SEQ ID NO: 33 - DNA, 74 bp, Artificial Sequence
  primer
cggatggcgcgccttatcattaagcgtaatctggaacatcgtatgggtacatctcgaggcagccggatcctttg

SEQ ID NO: 34 - DNA, 31 bp, Artificial Sequence
  primer
ttgatccagtttctttttctacgcctggccc

SEQ ID NO: 35 - DNA, 31 bp, Artificial Sequence
  primer
ttgatccagtttctttttggccgcctggccc

SEQ ID NO: 36 - DNA, 19 bp, Artificial Sequence
  primer
aaaaagaaactggatcaag

SEQ ID NO: 37 - protein, 16 aa, Artificial Sequence
  tryptic peptide derived from sfGFP
LQTNKNSHNVYITADK

SEQ ID NO: 38 - protein, 15 aa, Artificial Sequence
  a tryptic peptide derived from mCherry-EGFP-HA
AQASPWHKQLAMVSK

SEQ ID NO: 39 - protein, 16 aa, Artificial Sequence
  NLS of nucleoplasmin
KRPAATKKAGQAKKKK

SEQ ID NO: 40 - protein, 16 aa, Artificial Sequence
  NLS of p53
KRALPNNTSSSPQPKK

(19) United States
(12) Patent Application Publication
(10) Pub. No.: US 2013/0005019 A1
(43) Pub. Date: Jan. 3, 2013

(54) GENETICALLY ENCODED PHOTO CONTROL

(75) Inventors: Jason Chin, Cambridge (GB); Duy P. Nguyen, Cambridge (GB); Arnaud Gautier, Cambridge (GB); Alexander Dieters, Raleigh, NC (US)

(73) Assignees: North Carolina State University, Raleigh, NC (US); Medical Research Council, Swindon (GB)

(21) Appl. No.: 13/582,703

(22) PCT Filed: Mar. 4, 2011
(86) PCT No.: PCT/GB2011/000304

§ 371 Date: Sep. 4, 2012

(30) Foreign Application Priority Data
Mar. 5, 2010 (GB) 1003719.0
Sep. 16, 2010 (GB) 1015570.3

(51) Int. Cl. C07D 317/62; C12N 9/00; C12N 9/12

(52) U.S. Cl. 435/194; 549/439; 435/183

(57) ABSTRACT

The invention relates to a caged lysine, wherein the caged lysine is according to Formula (I): or salts thereof. The invention further relates to polypeptides comprising a caged lysine, and to methods of making same. The invention further relates to tRNA synthetases capable of charging tRNA with caged lysine.

### FIELD OF THE INVENTION

The invention relates to the provision of useful caging groups, their use in a method of site-specific introduction in proteins and the uses thereof.

### BACKGROUND OF THE INVENTION

Biologically active compounds may be protected with photo-removable protecting groups, altering important functionality in the molecule so as to block its biological efficacy. One mode of protecting such groups is known as caging. De-caging, for example by irradiation of the system, removes the protective (caging) group and restores the intrinsic property of the molecule.

Precise photochemical control of protein function can be achieved through the site-specific introduction of caging groups.¹² Chemical and enzymatic methods, including in vitro translation³ and chemical ligation⁴ have been used to photocage proteins in vitro. These methods have been extended to allow the introduction of caged proteins into cells by permeabilization⁵ or microinjection,⁶ but cellular delivery remains challenging.

Recently ortho-nitrobenzyl (ONB) caged versions of several amino acids have been genetically encoded in response to the amber stop codon.⁷⁸ The ONB group is stable under physiological conditions, but is readily removed with UV light of 250-365 nm.

The application of ONB disadvantageously uses the lower part of the UV light range of 250-365 nm for efficient photolysis which is toxic to cells because it leads to photoreactions of nucleic acids, destruction of disulphides and other cellular damage, which may occur when a simple ONB group is used to cage lysine.⁸

Lysine residues are key determinants for nuclear localization sequences,⁹ are the target of key post-translational modifications¹⁰ including ubiquitination, methylation, and acetylation, and are key residues in many important enzyme active sites. However, the application of ONB caging to lysine residues is further disadvantageous because the photolysis products of an ONB caged lysine residue leads to an undesired condensation of the ε-amino group of lysine.

Thus there is a problem in the art of providing an efficient caging molecule for lysine. It is a further problem to provide a method and/or a system to allow it to be incorporated site-specifically in proteins. It is a further problem to provide a method of producing said proteins whilst alleviating the present problems of cellular delivery of caged proteins.

### SUMMARY OF THE INVENTION

The present invention relates to a caged lysine molecule in which the caging group is induced by electron donating substituents to decage efficiently by irradiation with UV light above 340 nm.

The invention further relates to an orthogonal pyrollysyl-tRNA synthetase with mutations in up to 5 positions according to Table I wherein the mutations are present at residues M241, A267, Y271, L274 and C313, and the resulting orthogonal pyrollysyl-tRNA synthetase/tRNA pair therefrom. Another aspect of the invention relates to an in vitro method of incorporating the caged lysine amino acids according to the invention in a protein in a eukaryotic cell, wherein the method comprises the following steps:
i) introducing an amber codon at the desired site in, or replacing a specific codon in, the nucleotide sequence encoding the protein
ii) introducing the expression system as described herein into the cell
iii) growing the cells in a medium with the caged lysine as described herein present in the medium.

A still further aspect of the invention relates to the use of caged lysine amino acid according to the invention in determining or altering at least one property of a protein by UV light irradiation above 340 nm.

The invention relates to a caged lysine molecule in which the caging group is induced by electron donating substituents to decage efficiently at irradiation of UV light above 340 nm. Suitably the caging group decages efficiently at irradiation of UV light above 355 nm, preferably 365 nm.

Suitably the photolysis byproducts will not undergo condensation with the ε-amino group of lysine.

Suitably the caged lysine is according to Formula (I)

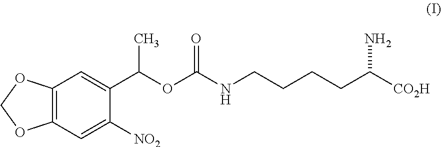 [SMILES: CC(OC(=O)NCCCC[C@H](N)C(=O)O)c1cc2c(cc1[N+](=O)[O-])OCO2]

or salts thereof.

In another aspect, the invention relates to a protein in which the caged lysine as described above has been incorporated into its amino acid sequence. Suitably the incorporation is site-specific. Suitably the incorporation of caged lysine is replacing a lysine amino acid. Suitably said replaced lysine amino acid was present in the naturally occurring sequence.

Suitably the protein is linked to a labelling molecule. Suitably the labelling molecule is a fluorescent protein.

In another aspect, the invention relates to a pyrollysyl-tRNA synthetase (an orthogonal pyrollysyl-tRNA synthetase) with mutation(s) in one to five positions according to Table I wherein the mutation(s) are present at one to five residues selected from M241, A267, Y271, L274 and C313. Suitably the orthogonal pyrollysyl-tRNA synthetase comprises four mutations, wherein the mutations are M241F, A267S, Y271C and L274M.

In another aspect, the invention relates to an orthogonal pyrollysyl-tRNA synthetase/tRNA pair wherein the orthogonal pyrollysyl-tRNA synthetase is an orthogonal pyrollysyl-tRNA synthetase as described above. Suitably the orthogonal tRNA is PyltRNACUA.

In another aspect, the invention relates to an expression system in eukaryotic cells for expressing orthogonal pyrollysyl-tRNA synthetase/tRNA pair as described above which comprises:

a nucleic acid such as a plasmid where PyltRNA_{CUA} expression is under the control of a U6 promoter downstream of a CMV enhancer
a nucleic acid such as a plasmid comprising the orthogonal pyrollysyl-tRNA synthetase as described above under control of a CMV enhancer.

An in vitro method of incorporating a caged lysine amino acid as described above into a protein in a cell, wherein the method comprises the following steps:

introducing, or replacing a specific codon with, an orthogonal codon such as an amber codon at the desired site in a nucleotide sequence encoding the protein
introducing the expression system as described above into the cell growing the cells in a medium with the caged lysine as described above present in the medium.

Suitably the amber codon replaces a codon for lysine in the nucleotide sequence encoding the protein.

In another aspect, the invention relates to a caged lysine amino acid as described above for use in determining at least one property of a protein by UV light irradiation above 340 nm.

In another aspect, the invention relates to a caged lysine amino acid as described above for use in altering at least one property of a protein by UV light irradiation above 340 nm.

In another aspect, the invention relates to a caged lysine amino acid as described above, wherein the altering of the at least one property allows measurement of the kinetics of the biological effect that result therefrom.

In another aspect, the invention relates to a caged lysine amino acid as described above, wherein the at least one property of the protein is the localisation of the protein in a eukaryotic cell.

Suitably the protein is in a eukaryotic cell.

Suitably the protein is in a human body.

Suitably the protein is in vitro.

The invention is now described by numbered paragraphs:
Paragraph 1. A caged lysine, wherein the caged lysine is according to Formula (I)

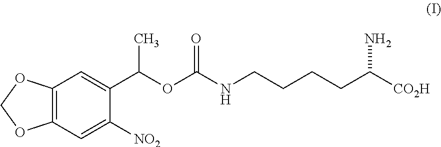 [SMILES: CC(OC(=O)NCCCC[C@H](N)C(=O)O)c1cc2c(cc1[N+](=O)[O-])OCO2]

or salts thereof.
Paragraph 2. A polypeptide comprising a caged lysine according to paragraph 1.
Paragraph 3. A polypeptide according to paragraph 2 wherein said caged lysine is present at a position in the polypeptide corresponding to a lysine residue in the wild type polypeptide.
Paragraph 4. A polypeptide according to paragraph 2 or paragraph 3 which is a nucleotide triphosphate binding protein.
Paragraph 5. A polypeptide according to paragraph 4 which is a kinase.
Paragraph 6. A polypeptide according to paragraph 5 wherein the caged lysine is present in the catalytic site of said kinase.
Thus the invention provides a photoactivatable kinase. The invention also relates to a method of photoactivating a kinase comprising decaging a caged lysine residue in the catalytic domain of said kinase.
Suitably the caged lysine is present at the conserved lysine residue of the catalytic site of a kinase, such as a residue corresponding to K97 of MEK.
Suitably the kinase is a member of a MAP kinase cascade. Suitably the kinase is a MEK (MAPKK).
Paragraph 7. A polypeptide according to paragraph 6 wherein decaging of the lysine permits kinase activity of said polypeptide.
Paragraph 8. A method of making a polypeptide comprising a caged lysine according to paragraph 1, said method comprising arranging for the translation of a RNA encoding said polypeptide, wherein said RNA comprises an orthogonal codon,
wherein said translation is carried out in the presence of tRNA recognising said orthogonal codon and capable of being charged with caged lysine according to paragraph 1, and in the presence of a tRNA synthetase capable of charging said tRNA with caged lysine according to paragraph 1, and in the presence of caged lysine according to paragraph 1.
Paragraph 9. A method according to paragraph 8 wherein the tRNA synthetase comprises pyrollysyl-tRNA synthetase with mutations relative to the wild type sequence in one to five positions according to Table I wherein the mutation(s) are present at positions corresponding to one to five residues selected from M241, A267, Y271, L274 and C313
Paragraph 10. A method according to paragraph 9 wherein the tRNA synthetase comprises four mutations, wherein the mutations are M241F, A267S, Y271C and L274M
Paragraph 11. A method according to any of paragraphs 8 to 10 wherein the orthogonal codon is an amber codon (TAG).
Paragraph 12. A method according to paragraph 11 wherein the orthogonal tRNA is PyltRNA_{CUA}
Paragraph 13. A method of making a polypeptide comprising caged lysine according to paragraph 1, said method comprising modifying a nucleic acid encoding said polypeptide to provide an amber codon at one or more position(s) corresponding to the position(s) in said polypeptide where it is desired to incorporate caged lysine according to paragraph 1.
Paragraph 14. A method according to paragraph 13 wherein modifying said nucleic acid comprises mutating a codon for lysine to an amber codon (TAG).
Paragraph 15. A homogenous recombinant polypeptide according to paragraph 2, wherein said polypeptide is made by a method according to any of paragraphs 8 to 14.
Paragraph 16. A pyrollysyl-tRNA synthetase with mutations relative to the wild type sequence in one to five positions according to Table I wherein the mutation(s) are present at positions corresponding to one to five residues selected from M241, A267, Y271, L274 and C313.
Paragraph 17. The orthogonal pyrollysyl-tRNA synthetase according to paragraph 16, comprising four mutations, wherein the mutations are M241F, A267S, Y271C and L274M.
Paragraph 18. An orthogonal pyrollysyl-tRNA synthetase/tRNA pair wherein the orthogonal pyrollysyl-tRNA synthetase is an orthogonal pyrollysyl-tRNA synthetase according to paragraph 16 or 17 and
wherein the orthogonal tRNA is PyltRNA_{CUA}.

### DESCRIPTION OF THE DRAWINGS

The invention will now be described in relation to the drawings in which:

FIG. 1—¹H NMR spectrum of compound 4

FIG. 2—¹H NMR spectrum of compound 1

FIG. 3—A. anti His-tag immunoblot of cell extracts from E. coli cells expressing PCKRS/PyltRNA_{CUA} and myoglobin with an amber codon at position 4 (pMyo4TAGHis6) in the presence or absence of 1 mM photocaged lysine 1. B. Coomassie stained gel of Ni-NTA purified sfGFP-his6 from cells containing either the MbPylRS/PyltRNA_{CUA} pair and grown with ε-Boc-lysine (BocK) (1 mM), or the PCKRS/PyltRNA_{CUA} pair and grown with 1 (5 mM). The unnatural amino acid was introduced into sfGFP in response to an amber codon at position 145. The yield of sfGFP-his6 obtained by incorporation of 1 using the PCKRS/PyltRNA_{CUA} pair was 1 mg/L, which is comparable with the yield obtained with BocK, known to be efficiently incorporated using the MbPylRS/PyltRNA_{CUA} pair¹⁶. C. ESI-MS analysis of myoglobin produced by PCKRS/PyltRNA_{CUA} (with 2 mM 1) revealed a mass of 18634 Da (peak A; expected mass 18631.7 Da). A second peak corresponding to myoglobin with a free lysine is also detected (peak B; obtained mass 18396 Da, expected mass 18395.7 Da). Since genetic and protein expression experiments indicated that protein expression is amino acid dependent this peak may result from the decaging of the incorporated 1 during sample preparation, where we cannot exclude light. D. MS/MS fragmentation of tryptic peptide derived from sfGFP(145-1) (the peptide sequence is shown above the spectrum; MH⁺ peptide mass 2145.972 Da). The spectrum confirms the incorporation of 1 at codon 145. The fragmentation sites are illustrated above the spectrum. Fragments with asterisk (*) do not contain the caged group due to the use of a MALDI laser at 355 nm which decages the sample. E. ESI-MS analysis of myoglobin produced by PCKRS/PyltRNA_{CUA} (with 2 mM 1) after photolysis for 0 min, 1 min and 5 min with 365 nm light (A: caged protein mass 18633.0±1.8 Da, expected mass 18631.7 Da; B: uncaged protein mass 18395.4±0.7 Da, expected mass 18395.7).

FIGS. 4-1. Genetic incorporation of a photocaged lysine in mammalian cells. A. Photocaged lysine 1. B, C. The PCKRS/PyltRNA_{CUA} pair allows for the specific incorporation of 1 (1 mM) in response to an amber codon in HEK293 cells; B. Fluorescence confocal micrographs of HEK293 cells expressing mCherry-TAG-egfp-ha and PCKRS/PyltRNA_{CUA} without and with 1; C. Immunoblot (IB) of cells from B with anti-HA. D. mCherry-EGFP-HA incorporating 1 expressed in HEK293 cells was purified by anti-HA immunoprecipitation for subsequent MS/MS analysis. The spectrum of the MS/MS fragmentation of a tryptic peptide derived from the purified protein confirms the incorporation of 1 at the expected site. Fragments labeled with an asterisk (*) result from decaging of peptide fragments during the MS/MS.

FIG. 5—The PCKRS/PyltRNA_{CUA} pair allows the specific incorporation of 1 (1 mM) in response to an amber codon into proteins in HEK293 cells; HEK293 cells were transfected with mCherry-TAG-egfp-ha and PCKRS/PyltRNA_{CUA} in the presence or absence of 1 mM 1. Anti-HA, anti-DsRed and anti-Flag immunoblots of the experiment are shown. The anti-HA immunoblot shows the expression level of full-length mCherry-GFP-HA, the anti-Ds-Red immunoblot shows the relative amount of truncated protein, and the anti-flag immunoblot show the expression level of PCKRS possessing a N-terminal flag-tag. Control experiments where PCKRS is absent or/and PyltRNA_{CUA} is absent or is replaced by hTyrtRNA_{CUA} are also shown.

FIG. 6—The MbPylRS/PyltRNA_{CUA} and MmPylRS/PyltRNA_{CUA} pairs (MbPylRS is from M. barkeri and MmPylRS is form M. mazei) allow the specific incorporation of E-Boc-lysine (BocK) (2 mM) in response to an amber codon into proteins in HEK293 cells; A. Fluorescence confocal micrographs of HEK293 cells expressing mCherry-TAG-egfp-ha and MbPylRS/PyltRNA_{CUA} in the presence or absence of 2 mM BocK (green: EGFP fluorescence, red: mCherry fluorescence). B. Fluorescence confocal micrographs of HEK293 cells expressing mCherry-TAG-egfp-ha and MmPylRS/PyltRNA_{CUA} in the presence or absence of 2 mM BocK (green: EGFP fluorescence, red: mCherry fluorescence). C. anti-HA and anti-Flag immunoblots of the experiment shown in A. and B. The anti-flag immunoblot shows the expression level of MbPylRS and MmPylRS possessing a N-terminal flag-tag. Control experiments where PyltRNA_{CUA} is absent or is replaced by hTyrtRNA_{CUA} are also shown. Boc=tert-butyloxycarbonyl.

FIG. 7—Photo-control of protein localization. A. Bipartite nuclear localization signal (NLS) of nucleoplasmin: the lysine in bold was mutated to alanine (NLS-A) or replaced by an amber stop codon (NLS-*). B. The PCKRS/PyltRNA_{CUA} pair allows the specific incorporation of 1 (1 mM) in response to the amber codon in nls-*-gfp-ha (lanes 2 and 3). Controls: expression of ^{WT}NLS-GFP-HA (lane 1), NLS-A-GFP-HA (lane 5), expression of NLS-*-Y-GFP (Y incorporation using hTyr-tRNA_{CUA}) (lane 4), non-transfected cells (lane 6). C. Fluorescence confocal micrographs showing the cellular localization of the GFP fusions; photolysis: 1 s, 365 nm, 1.2 mW/cm². D. Ratio F(n/c) of the mean nuclear and cytoplasmic GFP fluorescence before and 4 min after photolysis in the case of NLS-*-1-GFP-HA (data represents mean±SD of 27 cells, see FIG. 10 for representative examples). E. Kinetic analysis of the nuclear import process: the graph shows the normalized F(n/c) in function of time (mean±SD of 4 cells). A half-time of 20 s was determined. Scale bars 10 μm.

FIG. 8—Photocontrol of p53 localization. A. Bipartite nuclear localization signal of p53 (NLSₚ₅₃): the lysine K305 in bold was mutated to alanine (NLSₚ₅₃-K305A) or replaced by an amber stop codon (NLSₚ₅₃-K305A*). B. The PCKRS/PyltRNA_{CUA} pair allows the specific incorporation of 1 (1 mM) in response to the amber codon in p53-K305*-EGFP-HA in HEK293 cells (lane 2 and 3). Controls: expression of p53-EGFP-HA and p53-K305A-EGFP-HA (lane 1 and 5), expression of p53-K305*-Y-EGFP-HA (Y incorporation using hTyr-tRNA_{cu}A) (lane 4), non-transfected cells (lane 6). C. Fluorescence confocal micrographs showing the cellular localization of the EGFP fusions of wild-type p53, p53-K305A. D. Confocal micrographs showing the cellular localization of the EGFP fusions before and 50 min after photolysis (5 s; 365 nm; 1.2 mW/cm²). E. Ratio F(n/c) of the mean nuclear and cytoplasmic EGFP fluorescence before and 30 min after photolysis in the case of p53-K305*-1-EGFP-HA (data represents mean±SD of 7 cells). Scale bars 10 μm.

FIG. 9—A. Fluorescence confocal micrographs showing the cellular localization of the EGFP fusions of wild-type p53, p53-K305A, p53-K305*-Y (Y incorporation using hTyr-tRNA_{CUA}), p53-K305*-BocK (BocK incorporation using MbPylRS/PyltRNA_{CUA}) and p53-K305*-1 (incorporation of 1 using PCKRS/PyltRNA_{CUA}). B. p53-K305*-BocK localization before and 50 min after photolysis (5 s; 365 nm; 1.2 mW/cm²). C. Examples of p53-K305*-1-EGFP relocalization after photolysis (5 s; 365 nm; 1.2 mW/cm²). The time in minutes after photolysis is indicated on each frame. A 16-color scale is used to show the EGFP fluorescence. D. Kinetic analysis of p53 relocalization. The ratio F(n/c) of the mean nuclear and cytoplasmic GFP fluorescence is given in function of time for two different examples. Scale bars indicate 10 μm.

FIG. 10—Representative confocal micrographs showing the cellular localization of NLS*1-GFP fusions (incorporation of 1 using PCKRS/PyltRNAcuA) before and 4 min after photolysis (1-2 s; 365 nm; 1.2 mW/cm²). Scale bars indicate 10 μM.

FIG. 11 Maps of the main plasmids used

FIG. 12 shows a caged lysine and an application of the invention.

FIG. 13 shows alternative caged lysines applicable in the invention.

FIG. 14. Isolating a sub-network in MAP kinase signalling via genetically encoding of a photocaged lysine in the MEK1 active site. (a) Schematic of the MAP kinase signaling pathway and its photo-activable sub-network. (b) Caging a near-universally conserved lysine in the MEK1 active site inactivates the enzyme by sterically blocking ATP binding. Decaging with light rapidly removes the caging group and activates the kinase (figures created using Pymol and MEK1 structure PDB: 1S9J). (c) Structure of the photo-caged lysine 1, that can be genetically encoded by the PCKRS/tRNA_{CUA} pair, allowing the incorporation of 1 into proteins in response to an amber codon.

FIG. 15. Specific phosphorylation and activation of ERK2 upon photo-activation of the caged MEK1. (a) HEK293ET cells co-transfected with plasmids encoding PCKRS, pyrrolysyl tRNA_{CUA}, C-MEK1-ΔN-HA and EGFP-ERK2 (either TEY, lanes 7 and 8; or AAA, lanes 9 and 10) were grown in medium supplemented with 2 mM of amino acid 1 (lanes 8 and 10) or without (lanes 7 and 9) for 24 h. As controls, cells were transfected with plasmids encoding PCKRS, EGFP-ERK2 (TEY or AAA) and either: pyrrolysyl tRNA_{CUA} and A-MEK1-ΔN-HA (lanes 1 and 2); or pyrrolysyl tRNA_{CUA} and D-MEK1-ΔN-HA (lanes 3 and 4); or tyrosine tRNA^{Tyr}_{CUA} and C-MEK1-ΔN-HA (lanes 5 and 6, the incorporation of Tyr in response to the amber codon in C-MEK1-ΔN-HA gene via the use of the amber suppressor tyrosine tRNA^{Tyr}_{CUA} leads to an inactive MEK1 named D*-MEK1-ΔN-HA). (b) HEK293ET cells co-transfected with plasmids encoding PCKRS, pyrrolysyl tRNA_{CUA}, EGFP-ERK2 and either A-MEK1-ΔN-HA (lane 2), or D-MEK1-ΔN-HA (lanes 3-6), or C-MEK1-ΔN-HA (lanes 7-10) were grown in medium supplemented with 2 mM of 1 and 0.1% FBS for 24 h. Cells expressing D-MEK1-ΔN-HA and C-MEK1-ΔN-HA were illuminated with a 365 nm LED lamp for 60 s. Cells were lysed 1, 10 and 60 min after illumination. (c) HEK293ET cells co-transfected with plasmids encoding PCKRS, pyrrolysyl tRNA_{CUA}, EGFP-ERK2 and either A-MEK1-ΔN-HA (lane 2), or D-MEK1-ΔN-HA (lanes 3, 5, 6, 9, 10, 13, 14, 17, 18) or C-MEK1-ΔN-HA (lanes 4, 7, 8, 11, 12, 15, 16, 19, 20) were grown in medium supplemented with 2 mM of amino acid 1 and 0.1% FBS for 24 h. Cells expressing D-MEK1-ΔN-HA and C-MEK1-ΔN-HA were illuminated with a 365 nm LED lamp for 5 s (lanes 5-8), 15 s (lanes 9-12), 30 s (lanes 13-16) and 60 s (lanes 17-20). Cells were lysed 1 and 10 min after illumination. (d) HEK293ET cells co-transfected with plasmids encoding PCKRS, pyrrolysyl tRNA_{CUA}, EGFP-ERK2 and either C-MEK1-ΔN-HA (lanes 1-4), or D-MEK1-ΔN-HA (lanes 5-8) or A-MEK1-ΔN-HA (lanes 9-12) were grown in medium supplemented with 2 mM of amino acid 1 and 0.1% FBS for 24 h. Before illumination, cells were incubated with 0 or 10 μM of U0126 for 30 min. When indicated, cells were illuminated for 60 s with a 365 nm LED lamp. Cells were lysed 10 min after illumination. (a-d) Cell lysates were resolved by SDS-PAGE, followed by immunoblotting (IB) with the indicated antibodies.

FIG. 16 EGFP-ERK2 nuclear translocation upon EGF stimulation. (a) Montage showing EGFP-ERK2 sub-cellular fluorescence at different time points after activation of co-expressed wt-MEK1 by addition of 100 ng/ml EGF. Scale bars represent 5 μm. (b) The graph shows the normalized F(n/c) as a function of time after activation (mean±SD of seven representative cells). (c) The graph shows F(n/c) of seven independent experiments as a function of time after activation. (d) The graph shows normalized F(n/c) of seven independent experiments as a function of time after activation.

FIG. 17. Nuclear translocation of EGFP-ERK2 upon photo-activation of caged MEK1. (a) HEK293ET cells co-transfected with plasmids encoding PCKRS, pyrrolysine tRNA_{CUA}, and either C-MEK1-DD/EGFP-ERK2-TEY (cases 1 and 2), or D-MEK1-DD/EGFP-ERK2-TEY (case 3), or C-MEK1-DD/EGFP-ERK2-AAA (case 4) were grown in medium supplemented with 2 mM of amino acid 1 and 0.1% FBS for 24 h. In case 2, cells were pre-incubated with 10 μM of U0126. EGFP fluorescence of a representative cell before and 10 min after illumination (2 s, 365 nm, 1 mW/cm²) is shown in each case. The diagrams show the fluorescence intensity along the dotted lines before (black) and after (grey) illumination. Scale bars represent 10 μm. (b) Quantitative analysis of EGFP-ERK2 nuclear translocation. The graph on the left shows the ratio F(n/c) of the mean nuclear and cytoplasmic EGFP fluorescence before (white bars) and 10 min after illumination (black bars) in the cases shown in (a). For each case, mean±standard deviation (SD) of ten representative cells is shown. The graph on the right shows the difference of F(n/c) before and 10 min after illumination (ΔF(n/c)=F(n/c)_{after}−F(n/c)_{before}) in the cases shown in (a). For each case, data from ten representative cells are represented as box-and-whisker plot (the ends of the whiskers represent the minimum and maximum of all the data).

FIG. 18. Kinetics of EGFP-ERK2 nuclear translocation upon photo-activation of the caged MEK1. (a) Montage showing EGFP-ERK2 sub-cellular fluorescence at different time points after photo-activation (2 s, 365 nm, 1 mW/cm²) of co-expressed C-MEK1-DD. Scale bars represent 5 μm. (b) The graph shows the normalized F(n/c) as a function of time after photo-activation (mean±SD of ten representative cells). In grey line is shown as a comparison the normalized F(n/c) observed when cells were stimulated with EGF and presented in FIG. 16b. (c) The graph shows F(n/c) of ten experiments as a function of time after activation. (d) The graph shows normalized F(n/c) of ten independent experiments as a function of time after activation. (e,f) Comparison of the cell-to-cell variability observed in EGFP-ERK2 nuclear translocation upon stimulation with EGF (data shown on FIG. 16b-d, n=7 cells) and upon photo-activation of C-MEK1-DD (data shown in b-d, n=10 cells). The two graphs show respectively (e) the half-time of the translocation process upon activation (t_{1/2}) and (f) the change in F(n/c) observed (ΔF(n/c)=max(F(n/c))−min(F(n/c))). The data are represented as box-and-whisker plot (the ends of the whiskers represent the minimum and maximum of all the data). (g) Montage showing representative EGFP-ERK2 sub-cellular fluorescence at different time points early after photo-activation (2 s, 365 nm, 1 mW/cm²) of co-expressed C-MEK1-DD (see also Movie S1). Scale bars represent 10 μm. (h) Kinetics of translocation early after photo-activation. Normalized F(n/c) as a function of time after photo-activation (mean±SD of ten representative cells) is shown. Data were fitted with a sigmoidal function.

FIG. 19. ERK2 nucleocytoplasmic shuttling. (a) HEK293ET cells co-expressing C-MEK1-DD and EGFP-ERK2 were illuminated (2 s, 365 nm, 1 mW/cm²), then 8 minutes after illumination, U0126 (10 μM) was added to block the activity of photoactivated C-MEK1-DD and unveil EGFP-ERK2 efflux from the nucleus. The bottom montage shows representative EGFP-ERK2 sub-cellular fluorescence at different times after illumination and post-illumination blockage with U0126. The top montage shows as a reference the EGFP-ERK2 sub-cellular fluorescence at different times after photo-activation without addition of U0126. Scale bars represent 5 μm. (b) The graph presents the normalized F(n/c) as a function of time after illumination (mean±SD of ten representative cells). The arrow indicates the time when U0126 was added. As a comparison, the normalized F(n/c) without addition of the inhibitor is presented in FIG. 18b is plotted as a grey line.

FIG. 20. (a) Montage showing EGFP-ERK2 (top) and EGFP-ERK2Δ4 (bottom) sub-cellular fluorescence at different time points after photo-activation (2 s, 365 nm, 1 mW/cm²) of co-expressed C-MEK1-DD. Scale bars represent 5 μm. (b) The graph shows the kinetics of nuclear translocation of EGFP-ERK2A4 upon photo-activation of C-MEK1-DD (mean±SD for ten representative cells). In grey line is shown as a comparison the kinetics of nuclear translocation of EGFP-ERK2 shown in FIG. 18b. (c) The plot shows the maximum of F(n/c) from the experiments shown in (a) (mean±SD for ten representative cells). (d,e) HEK293ET cells co-expressing C-MEK1-DD and either EGFP-ERK2 or EGFP-ERK204 were illuminated with a LED lamp for 1 minute and lysed after 1, 5, 10, 15, 20 and 30 minutes. (d) Cell lysates were resolved by SDS-PAGE, followed by immunoblotting (IB) with the indicated antibodies. (e) The phosphorylation of the EGPF-ERK2 mutants observed in (d) was quantified and normalized by their expression level, and plotted as a function of time (representative data from three independent data sets).

FIG. 21. (a) HEK293ET cells co-transfected with plasmids encoding PCKRS, pyrrolysine tRNA_{CUA} and C-MEK1-ΔN-HA (lanes 3 and 4) were grown in medium supplemented with 1 mM 1 (lane 4) or without (lane 3) for 24 h. As controls, cells were co-transfected with plasmids encoding PCKRS and either: pyrrolysine tRNA_{CUA} and A-MEK1-ΔN-HA (lane 1); or pyrrolysine tRNA_{CUA} and D-MEK1-ΔN-HA (lane 2); or tyrosine tRNA_{Tyr CUA} and C-MEK1-ΔN-HA (lane 5; the incorporation of Tyr in response to the amber codon in C-MEK1-ΔN-HA gene via the use of the amber suppressor tyrosine tRNA_{Tyr CUA} leads to an inactive dead MEK1 named D*-MEK1-ΔN-HA). Cell lysates were resolved by SDS-PAGE, followed by immunoblotting (IB) with the indicated antibodies. (b) Immunoblot comparing the expression level of the different MEK1-ΔN-HA mutants with endogenous MEK.

FIG. 22. Cells were co-transfected with plasmids encoding PCKRS, EGFP-ERK2 and either: pyrrolysine tRNA_{CUA} and A-MEK1-ΔN-HA (lanes 1 and 2); or pyrrolysine tRNA_{CUA} and D-MEK1-ΔN-HA (lanes 3 and 4); or pyrrolysine tRNA_{CUA} only (lanes 5 and 6); or tyrosine tRNA_{Tyr CUA} and C-MEK1-ΔN-HA (lanes 7 and 8; the incorporation of Tyr in response to the amber codon in C-MEK1-ΔN-HA gene via the use of the amber suppressor tyrosine tRNA_{Tyr CUA} leads to an inactive dead MEK1 named D*-MEK1-AN-HA); or pyrrolysine tRNA_{CUA} and C-MEK1-ΔN-HA (lanes 9 and 10). Lanes 11 and 12 show mock non-transfected cells. After transfection, HEK293ET cells were grown in medium supplemented with 2 mM 1 and 0.1% FBS for 24 h. When indicated, cells were illuminated with a 365 nm LED lamp for 60 s, and lysed 60 min after illumination. Cell lysates were resolved by SDS-PAGE, followed by immunoblotting (IB) with the indicated antibodies.

FIG. 23. (a) HEK293ET cells co-transfected with plasmids encoding PCKRS, pyrrolysine tRNA_{CUA} and C-MEK1-DD-HA (lanes 3 and 4) were grown in medium supplemented with 2 mM 1 (lane 4) or without (lane 3) for 24 h. As controls, cells were co-transfected with plasmids encoding PCKRS and either: pyrrolysine tRNA_{CUA} and A-MEK1-DD-HA (lane 1); or pyrrolysine tRNA_{CUA} and D-MEK1-DD-HA (lane 2); or tyrosine tRNA_{Tyr CUA} and C-MEK1-DD-HA (lane 5; the incorporation of Tyr in response to the amber codon in C-MEK1-DD-HA gene via the use of the amber suppressor tyrosine tRNA_{Tyr CUA} leads to an inactive dead MEK1 named D*-MEK1-DD-HA). (b) HEK293ET cells co-transfected with plasmids encoding PCKRS, pyrrolysine tRNA_{CUA}, EGFP-ERK2 and either A-MEK1-ΔN-HA (lane 1), or D-MEK1-ΔN-HA (lanes 2-3) or C-MEK1-ΔN-HA (lanes 4-5), or A-MEK1-DD-HA (lane 6), or D-MEK1-DD-HA (lanes 7-8) or C-MEK1-DD-HA (lanes 9-10) were grown in medium supplemented with 2 mM 1 and 0.1% FBS for 24 h. When indicated, cells expressing D-MEK1-ΔN or DD)-HA and C-MEK1-(ΔN or DD)-HA were illuminated with a 365 nm LED lamp for 60 s. Cells were lysed 10 min after illumination. (a-b) Cell lysates were resolved by SDS-PAGE, followed by immunoblotting (IB) with the indicated antibodies.

### DETAILED DESCRIPTION OF THE INVENTION

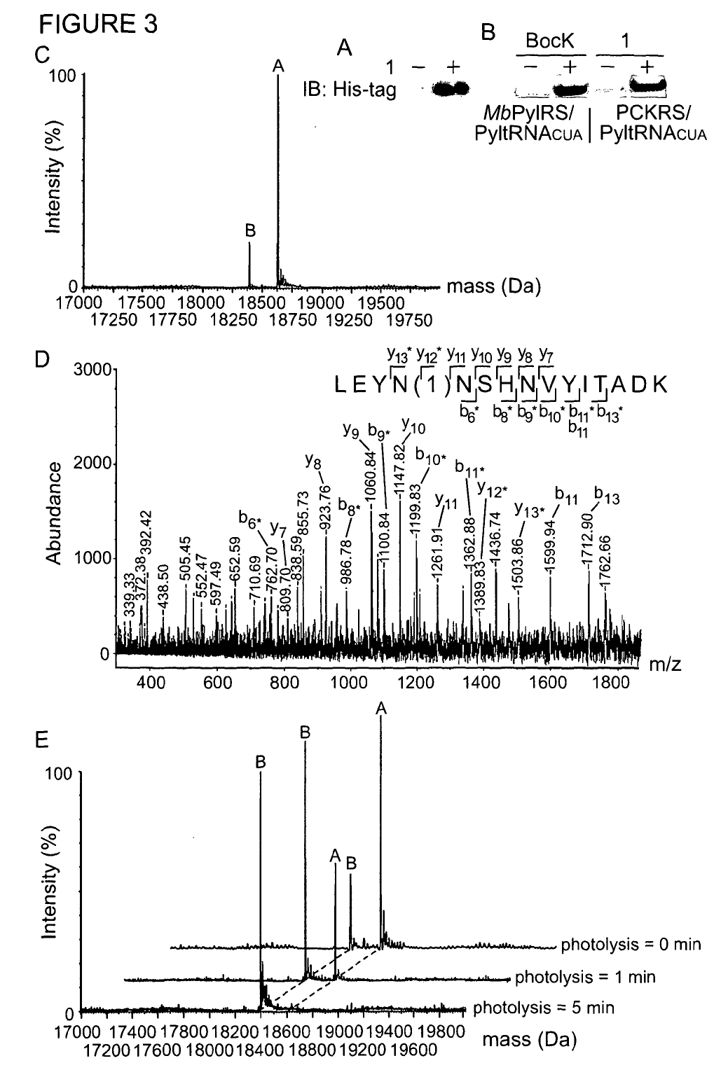

The present invention relates to a caged lysine molecule in which the caging group is induced by electron-donating substituents to decage efficiently at irradiation of UV light above 340 nm. The effect of the electron donation to the caging group allows the caging group to be decaged efficiently when irradiated with light above 340 nm. Preferably the UV irradiation is above 355 nm, preferably between 360 and 370 nm, even more preferably about 365 nm. It is clear to the person skilled in the art that the advantage with respect to other caging molecules is the efficiency of photolysis of the caged molecule, when irradiated at these higher UV wavelengths. As shown in FIG. 3 and Example 3, after 5 minutes, essentially the entire population of caged protein is de-caged by UV irradiation at 365 nm.

It is further preferable if the caging group is constructed so that upon photolysis, the by products of the photolysis do not react in a condensation reaction with the e-amino group of lysine. A preferred embodiment is when the caged lysine according to the present invention is according to Formula (I)

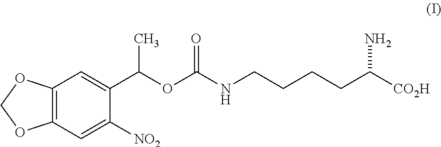 [SMILES: CC(OC(=O)NCCCC[C@H](N)C(=O)O)c1cc2c(cc1[N+](=O)[O-])OCO2]

or salts thereof.

Another aspect of the invention is the caged lysine as described above when incorporated into the amino acid sequence of a protein. The advantage, as discussed below, is that it allows the determination and/or alteration of a specific property in a protein. It is preferable that the incorporation be site-specific, as this advantageously allows determination/alteration of a specific property of the protein due to the presence of the caged lysine in a specific point of the protein.

The site-specific incorporation of the caged lysine amino acid may be at any point in the polypeptide sequence. This is typically accomplished by site specific mutation of the nucleotide sequence of a nucleic acid encoding the polypeptide of interest, followed by transcription (if necessary) and translation of that nucleic acid into polypeptide. The incorporation may be by replacement of an existing codon or may be by insertion of a codon. Typically the codon used to specify the caged lysine will be the amber codon TAG (CUA). However, of course if a tRNA synthetase-tRNA pair used for incorporation comprises a tRNA recognising a different codon (or a quadruplet codon), then the corresponding cognate codon of that tRNA synthetase-tRNA pair will be used in place of the amber codon. The amber codon is a preferred example of a suitable orthogonal codon by which genetic incorporation may be easily achieved, but is not intended to limit or to exclude the use of other codon(s) provided that a suitable system for charging the cognate tRNA of any such other codon(s) can be employed.

It is further preferable that the site-specific incorporation of the caged lysine amino acid in the amino acid sequence of the protein be as replacement of a lysine residue present in the wild-type sequence of the protein. The advantage of said protein is that it allows empirical determination the intrinsic properties of that lysine residue and therefore the biological effect(s) of the protein mediated by (or influenced by) that lysine once the irradiation and resulting de-caging occurs.

In one preferred embodiment, the protein according to the invention as described above is further linked to a labelling molecule. The labelling molecule can be any molecule which a person skilled in the art can use under experimental circumstances to determine some biologically relevant property or function of the protein. Some examples of such molecules are radioactive elements, fluorescent or luminescent markers. The method of linking the protein to the labelling molecule depends entirely on the type of labelling molecule used and the choice is well within the person skilled in the art's expertise. In a preferred example of said system, the labelling molecule is a fluorescent protein, such as GFP, fused to the C-terminal of the protein with the caged lysine incorporated in it. Said example is preferred as the method of linking the protein is easily achieved by incorporating a nucleotide sequence encoding the GFP protein into a plasmid which encodes the protein with the caged amino acid. In said preferred example, the resulting protein expressed in the cell is easily visualised.

Another aspect of the invention is a method, such as an in vitro method, of incorporating the caged lysine amino acids genetically and site-specifically into the protein of choice, suitably in a eukaryotic cell. One advantage of incorporating it genetically by said method is that it obviates the need to deliver the proteins comprising the caged amino acid into a cell once formed, since in this embodiment they may be synthesised directly in the target cell. The method comprises the following steps:
i) introducing, or replacing a specific codon with, an orthogonal codon such as an amber codon at the desired site in the nucleotide sequence encoding the protein
ii) introducing an expression system of orthogonal pyrollysyl-tRNA synthetase/tRNA pair in the cell
iii) growing the cells in a medium with the caged lysine according to the invention.

Step (i) entails or replacing a specific codon with an orthogonal codon such as an amber codon at the desired site in the genetic sequence of the protein. This can be achieved by simply introducing a construct, such as a plasmid, with the nucleotide sequence encoding the protein, wherein the site where the caged lysine is desired to be introduced/replaced is altered to comprise an orthogonal codon such as an amber codon. This is well within the person skilled in the art's ability and examples of such are given here below.

Step (ii) requires an orthogonal expression system to specifically incorporate the caged lysine amino acid at the desired location (e.g. the amber codon). Thus a specific orthogonal tRNA synthetase such as an orthogonal pyrollysyl-tRNA synthetase and a specific corresponding orthogonal tRNA pair which are together capable of charging said tRNA with the caged lysine are required.

Thus another aspect of the invention is the provision of an orthogonal tRNA synthetase such as a pyrollysyl-tRNA synthetase for the caged lysine according to the invention. Said orthogonal pyrollysyl-tRNA synthetase are suitably wild-type Pyrollysyl-tRNA synthetase with mutation(s) in up to 5 positions as defined in Table I wherein the mutation(s) are present at residues M241, A267, Y271, L274 and C313. In a preferred embodiment, the orthogonal pyrollysyl-tRNA synthetase is clone 7 of Table I, i.e. wherein the mutations are M241F, A267S, Y271C and L274M, which has the advantage of being found to be the most efficient synthetase clone as defined in Table I.

The orthogonal pyrollysyl-tRNA synthetase according to the invention needs to be associated with an orthogonal tRNA to constitute an expression system to be able to execute step (ii) of the method above. The use of PylT, the gene encoding PyltRNA_{CUA}, lacks the consensus internal RNA polymerase III promoter sequences found in eukaryotic tRNAs and is well known in the art as an orthogonal tRNA system to be used in an orthogonal pyrollysyl-tRNA synthetase/tRNA pair¹⁴. It requires an external promoter for transcription. Preferably tRNA expression is under the control of a U6 promoter downstream of a CMV enhancer,¹⁵ enabling efficient transcription of PylT.

Thus a preferred expression system to be used in step (ii) of the method above comprises:
a. a plasmid where PyltRNACUA expression is under the control, of a U6 promoter downstream of a CMV enhancer
b. a plasmid comprising the orthogonal pyrollysyl-tRNA synthetase as described herein under control of a CMV enhancer.

In another aspect of the invention, the caged lysine according to the invention can be used for determining or altering at least one property of a protein by UV light irradiation above 340 nm. Preferably the irradiation is above 355 nm, more preferably between 360 and 370 nm, even more preferably about 365 nm.

The advantage of such uses is that the mode of switching from caged to de-caged is efficiently achieved, both in the sense of time and percentage of protein with caged lysines being de-caged, and that such a system is non-invasive and not toxic to cells.

Advantageously such a system can be used for determination or alteration of at least one property of a protein in a eukaryotic cell, even within a human body.

Said at least one property of the protein may be a biochemical property of the protein which is present in the wild-type protein and not present when the caged amino acid is present. This may be the sole biological function of the protein, or may be one or more of several properties of the protein. An example where the property is not the sole biological function of the protein is the NLS sequences present in a tumour suppressor p53. The property and its effects on the protein can vary according to the size, shape of the protein and also importantly the position of incorporation of the caged lysine in the polypeptide chain. Thus when used for determination upon de-caging by photolysis, the invention enables the operator to study how the property impeded by the caged lysine residue affects the biological effect of the protein upon de-caging. In its use in altering a property of a protein, the biological effects resulting from the alteration may be known and therefore studied, or may be unknown in which case the invention may be advantageously applied to the determination or inference of such properties. An example of application of the invention to a known property would be the desire to release a caged lysine placed in a localisation sequence so as to allow the uncaged sequence to then localise the protein in the appropriate cellular compartment, thereby permitting kinetic studies or other observations to be carried out.

It is preferable for the caged lysine to replace a lysine present in the wild-type protein. This is preferable as it allows determination, through de-caging, of the intrinsic function of the protein in the cell as the protein reverts to its wild-type structure on de-caging.

When being used for alteration, it is assumed that the biological effects resulting from the alteration are known and/or desired. Such use of the caged lysine as alternator (switch) can be actuated to study the kinetics of proteins resulting from the de-caging. One example is when the protein folding is disturbed by the presence of a caged lysine. In such a case, the de-caging of the caged amino acid would allow protein folding to occur again, thus allowing one to measure the kinetics of protein folding that result from the de-caging. Another example is the incorporation of a caged lysine amino acid in a localisation sequence. This would disrupt the proper localisation of the protein until the de-caging, allowing one to measure the kinetics of protein localisation. Thus, embodiments of the invention in which the properties of the protein of interest are altered by decaging are sometimes referred to as ‘switching’ or ‘alternation’ (i.e. moving to an alternate form of the protein in the decaged state).

It is further contemplated that the use described here above regarding the altering (alternating) of at least one property of a protein by UV light irradiation above 340 nm may be used for therapeutic purposes. The alternation by de-caging may allow a protein, which previously was undesired to be localised/have a certain function/be fully folded, to then localise/have a certain function/be fully folded and thus have a certain therapeutic function. Examples of proteins where such a situation may occur are membrane proteins, especially expression of known cluster of differentiation proteins or for example antibodies or proteins belonging to the complement immune system.

### Caged Lysine Species

Alternative caged lysines other than that of Formula I may be used in the invention. Examples of useful caged lysine compounds are shown in FIG. 13.

Possible compounds are reviewed in Mayer et al. Angew. Chem. Int. Ed. 45, 4900-4921 (2006).

Compounds shown in FIG. 13 are specifically described in the following citations. The sections of these citations describing the compounds shown in FIG. 13 are specifically incorporated herein by reference, in particular for the details of structure or production of the corresponding compound(s) shown in FIG. 13:

compound 4: Momotake et al. Nat. Meth. 3, 35-40 (2006)

compound 5: Walbert et al. Helv. Chim. Acta 84, 1601-1611 (2001)

compound 6: Singh et al. Bioconjug. Chem. 13, 1286-1291 (2002)

compound 7: Furuta et al. Proc. Nat. Acad. Sci. 96, 1193-1200 (1999); Suzuki et al. Org. Lett. 5, 4867-4870 (2003); Hagen et al. ChemBioChem 4, 434-442 (2003).

compound 8: Fedoryak et al. Org. Lett. 4, 3419-3422 (2002).

compound 9: Park et al. JACS 119, 2453-2463 (1997); Zhang et al. JACS 121, 5625-5632 (1999); Conrad I I et al. Org Lett 2, 1545-1547 (2000).

compound 10: Atemnkeng et al. Org Lett 5, 4469-4471 (2003).

compound II: Klán et al. Photochem Photobiol Sci 1, 920-923 (2002); Klán et al. Org Lett 2, 1569-1571 (2000)

Most suitably the caged lysine is as shown in Formula I.

Reference Sequences

The Methanosarcina barkeri PylT gene encodes the MbtRNA_{CUA} tRNA.

The Methanosarcina barkeri PylS gene encodes the MbPylRS tRNA synthetase protein. When particular amino acid residues are referred to using numeric addresses, the numbering is taken using MbPylRS (Methanosarcina barkeri pyrrolysyl-tRNA synthetase) amino acid sequence as the reference sequence (i.e. as encoded by the publicly available wild type Methanosarcina barkeri PylS gene Accession number Q46E77):

MDKKPLDVLI SATGLWMSRT GTLHKIKHYE VSRSKIYIEM
ACGDHLVVNN SRSCRTARAF RHHKYRKTCK RCRVSDEDIN
NFLTRSTEGK TSVKVKVVSA PKVKKAMPKS VSRAPKPLEN
PVSAKASTDT SRSVPSPAKS TPNSPVPTSA PAPSLTRSQL
DRVEALLSPE DKISLNIAKP FRELESELVT RRKNDFQRLY
TNDREDYLGK LERDITKFFV DRDFLEIKSP ILIPAEYVER
MGINNDTELS KQIFRVDKNL CLRPMLAPTL YNYLRKLDRI
LPDPIKIFEV GPCYRKESDG KEHLEEFTMV NFCQMGSGCT
RENLESLIKE FLDYLEIDFE IVGDSCMVYG DTLDIMHGDL
ELSSAVVGPV PLDREWGIDK PWIGAGFGLE RLLKVMHGFK
NIKRASRSES YYNGISTNL

This is to be used as is well understood in the art to locate the residue of interest. This is not always a strict counting exercise—attention must be paid to the context. For example, if the protein of interest is of a slightly different length, then location of the correct residue in that sequence to corresponding to (for example) Y271 may require the sequences to be aligned and the equivalent or corresponding residue picked, rather than simply taking the 271st residue of the sequence of interest. This is well within the ambit of the skilled reader.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably a randomisation of said site is used, for example as described herein in connection with the evolution and adaptation of tRNA synthetase of the invention. As a default mutation, alanine (A) may be used. Suitably the mutations used at particular site(s) are as set out herein.

A fragment is suitably at least 10 amino acids in length, suitably at least 25 amino acids, suitably at least 50 amino acids, suitably at least 100 amino acids, suitably at least 200 amino acids, suitably at least 250 amino acids, suitably at least 300 amino acids, suitably at least 313 amino acids, or suitably the majority of the tRNA synthetase polypeptide of interest.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

The following non-limiting examples are illustrative of the present invention:

In all examples, the caged lysine according to Formula (I) is either denoted as such or as compound 1.

We teach photocaging of lysine to control protein localization, post-translational modification and enzymatic activity. The photochemical control of these important functions mediated by lysine residues in proteins has not previously been demonstrated in living cells. Here we synthesize 1, and evolve a pyrrolysyl-tRNA synthetase/tRNA pair to genetically encode the incorporation of this amino acid in response to an amber codon in mammalian cells. To exemplify the utility of this amino acid we cage the nuclear localization sequences (NLSs) of nucleoplasmin and the tumor suppressor p53 in human cells, thus mis-localizing the proteins in the cytosol. We trigger protein nuclear import with a pulse of light allowing us to directly quantify the kinetics of nuclear import.

### Example 1

### Synthesis of Cooed Lysine According to Formula I

The nitrobenzyl caged lysine 1 was prepared by reacting N⁺-Boc-lysine with the chloroformate 3 in a basic THF/H₂O solution at 0° C. providing 4 in 82% yield, followed by deprotection with TFA in CH₂Cl₂ in 95% yield (Scheme S1). The chloroformate 3 was generated through an acylation of the alcohol 3 (synthesized according to ref 19) with triphosgene in THF in the presence of Na₂CO₃, followed by evaporation of the volatiles and a direct reaction without further purification. The presence of Na₂CO₃ prevented dehydration of 2 to the corresponding styrene.

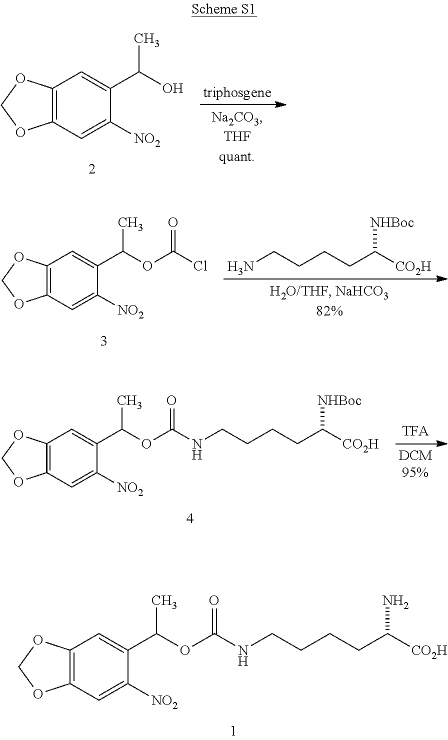 [SMILES: CC(O)c1cc2c(cc1[N+](=O)[O-])OCO2 ; CC(OC(=O)Cl)c1cc2c(cc1[N+](=O)[O-])OCO2 ; CC(C)(C)OC(=O)N[C@@H](CCCCN)C(=O)O ; CC(OC(=O)NCCCC[C@H](NC(=O)OC(C)(C)C)C(=O)O)c1cc2c(cc1[N+](=O)[O-])OCO2 ; CC(OC(=O)NCCCC[C@H](N)C(=O)O)c1cc2c(cc1[N+](=O)[O-])OCO2]

Synthetic Protocols

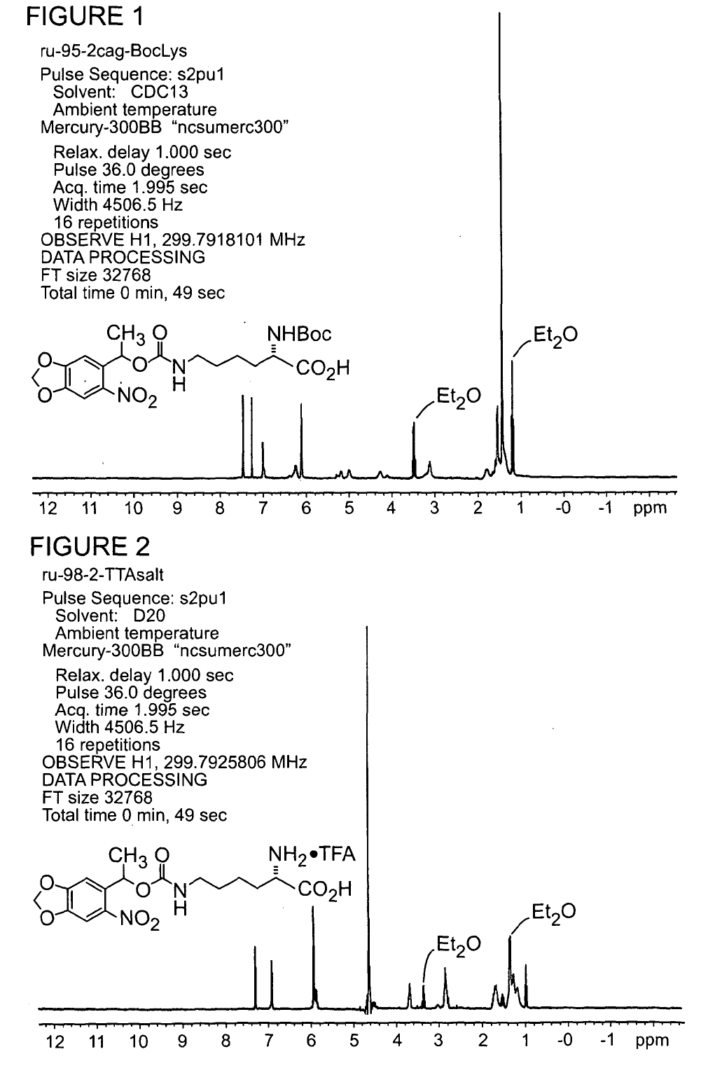

(2S)-2-(tert-Butoxycarbonylamino)-6-{[1-(6-nitrobenzo[d][1,3]dioxol-5-yl)ethoxy]carbonylamino}hexanoic acid (4). 1-(6-Nitrobenzo[d][1,3]dioxol-5-yl)ethanol (2) (500 mg, 2.36 mmol) was dissolved in THF (5 mL), containing Na₂CO₃ (247 mg, 2.36 mmol), and cooled to 0° C. To the solution was added triphosgene (701 mg, 2.36 mmol) and the reaction was kept stirring for 12 h at r.t. The reaction was filtered and the volatiles were subsequently evaporated without heating and the residue dried under vacuum, to give NPOC chloroformate 3 in quantitative conversion (644 mg, 2.36 mmol). To a solution of N^{ε}-Boc-lysine (500 mg, 2.02 mmol) in THF/1 M NaOH (aq.) (1:4 mixture, 8 mL total), at 0° C., was added NPOC-α-methyl chloroformate 3 (496 mg, 1.82 mmol). After the reaction was stirred for 12 h, at r.t., the aqueous layer was washed with Et₂O (5 mL) and subsequently acidified with ice-cold 1 M HCl (20 mL) to pH 1 and extracted with EtOAc (30 mL). The organic layer was dried over Na₂SO₄, filtered, and the volatiles were evaporated, affording 4 as a yellow foam in 82% yield (720 mg, 1.49 mmol). ¹H NMR (300 MHz, CDCl₃) δ=1.18-1.81 (m, 18H), 3.08 (br s, 2H), 4.23 (br s, 1H), 5.11-5.38 (m, 1H), 6.07 (s, 2H), 6.20-6.36 (m, 1H), 6.99 (s, 1H), 7.40 (s, 1H). ¹³C NMR (75 MHz, CHCl₃) δ=22.3, 22.6, 28.5, 29.4, 32.3, 40.8, 53.3, 69.1, 80.4, 103.3, 105.4, 105.8, 136.7, 141.5, 147.2, 152.6, 155.7, 156.1, 176.4. HRMS: m/z calcd for C₂₁H₂₉N₃O₁₀ [M+Na]⁺: 506.1745; found: 506.1748. (see FIG. 1 for ¹H NMR spectrum)

(2S)-2-Amino-6-{([1-(6-nitrobenzo[d][1,3]dioxol-5-yl)ethoxy]carbonylamino}hexanoic acid TFA salt (1). Compound 4 (720 mg, 1.49 mmol) was dissolved in DCM:TFA (1:1 mixture, 14 mL total) and the reaction was allowed to stir for 40 min. The volatiles were subsequently evaporated and the residue was redissolved in MeOH (5 mL) and precipitated into Et₂O (250 mL), giving 1 as a white solid in 95% yield (679 mg, 1.42 mmol). ¹H NMR (300 MHz, D₂O) δ=1.08-1.40 (m, 7H), 1.63-1.88 (m, 2H), 2.80-2.88 (m, 2H), 3.83-3.97 (m, 1H), 5.91-6.00 (m, 3H), 6.92 (s, 1H), 7.28 (s, 1H). ¹³C NMR (75 MHz, D₂O) δ=21.1, 21.7, 28.5, 29.5, 39.9, 52.7, 68.8, 103.6, 104.4, 105.7, 136.1, 140.6, 146.9, 152.7, 156.9, 171.9. HRMS: m/z calcd for C₁₅H₂₁N₃O₆ [M+H]⁺: 384.1402; found: 384.1403. (see FIG. 2 for ¹H NMR spectrum)

### Example 2

### Synthesis of an Orthogonal Pyrrolysyl-Trna Synthetase/tRNA Pair for Caged Lysine According to Formula (I)

To evolve the orthogonal MbPylRS/PyltRNA_{CUA} pair¹¹ for the incorporation of the caged lysine 1 in response to an amber codon, a library of 10⁸ mutants of MbPylRS was created in which 5 positions (M241, A267, Y271, L274, C313) in the binding pocket of the pyrrolysine ring were randomized to all possible amino acids.

pBKAcKRS3amp²⁰ was used as a template in the generation of a library of MbPylRS mutants. Three rounds of inverse PCR²¹ were performed to randomize codons for M241, A267, Y271, L274 and C313 to all 20 natural amino acids in this library. The following primers were used in each round of PCR reactions:

(round 1)
PyISM241f
(5′-GCGCAGGTCTCAGAACGTNNKGGCATTAACAACGACACCGAAC
TGAGCAAAC-3′)
and
PyISM241r
(5′-GCGCAGAGTAGGTCTCAGTTCCACATATTCCGCCGGAATCAGAA
TC-3′);
(round 2)
PyISAYLf
(5′-GCGCAGGTCTCAATGCTGN
NKCCGACCCTGNNKAACTATNNKCGTAAACTGGATCGTATTCTGCC
GGGC-3′)
and
PyISAYLr
(5′-GCGCAGAGTAGGTCTCAGCATCGGACGCAGGCACAGGTTTTTAT
C-3′);
(round 3)
PyISC313f
(5′-GCGCAGGAAAGGTCTCAAACTTTN
NKCAAATGGGCAGCGGCTGCACCCGTGAAAAC-3′)
and
PyISC313r
(5′-GCGCAGAGTAGGTCTCAAGTTAACCATGGTGAATTCTTCCAGGTGT
TCTTTG-3′).

The PCR product in each round was first digested with DpnI and BsaI, re-circularized by ligation and used to transform electrocompetent DH10B. The reisolated plasmids served as template for the next round of mutagenesis. Transformation of electro-competent DH10B with the ligation of the third round of mutagenesis produced 10⁸ transformants, covering the theoretical diversity of the library (2×10⁷) by more than 99%. Selection of mutants specific for 1 was carried out as described for the evolution of a synthetase specific for acetyl-lysine¹¹.

Three rounds of alternating positive and negative selection on this library in E. coli were performed, as previously described.^{11,12} Clones that survived the selection were transformed with a plasmid encoding the chloramphenicol resistance gene with an amber codon at a permissive position. The best clones allowed cells to survive on media containing up to 300 μg/ml chloramphenicol in the presence of 1 (1 mM), but did not survive at 50 μg/ml in the absence of 1. This demonstrates that the selected synthetases have a high specificity for 1, and do not incorporate any of the common 20 amino acids. The most active synthetase contained the mutations M241F, A267S, Y271C, and L274M with respect to wild-type MbPylRS. This synthetase was named Photocaged Lysyl-tRNA Synthetase (PCKRS) and was further characterized (see Table 1 for all isolated MbPylRS sequences).

### Example 3

### Demonstration of De-Caging Upon Irradiation with 365 Nm Light in Myoglobin In Vitro

1. Expression and Purification of Myoglobin

To express myoglobin with an incorporated unnatural amino acid, we transformed E. coli DH10B cells with pBKamp-PCKRS and pMyo4TAGPylT-his6. Cells were recovered in 1 mL of LB media for 1 h at 37° C., before incubation (16 h, 37° C., 250 r.p.m.) in 100 mL of LB containing ampicillin (100 μg/mL) and tetracycline (25 μg/mL). 20 mL of this overnight culture was used to inoculate 1 L of LB supplemented with ampicillin (50 μg/mL), to tetracycline (12 μg/mL) and 2 mM of 1. Cells were grown (37° C., 250 r.p.m.), and protein expression was induced at OD₆₀₀˜0.6, by addition of arabinose to a final concentration of 0.2%. After 3 h of induction, cells were harvested. Proteins were extracted by sonication at 4° C. The extract was clarified by centrifugation (20 min, 21,000 g, 4° C.), 300 μL of Ni²⁺-NTA beads (Qiagen) were added to the extract, the mixture was incubated with agitation for 1 h at 4° C. Beads were collected by centrifugation (10 min, 1000 g). The beads were twice resuspended in 50 mL wash buffer and spun down at 1000 g. Subsequently, the beads were resuspended in 20 ml of wash buffer and transferred to a column. Protein was eluted in 1 ml of wash buffer supplemented with 250 mM imidazole and was then re-buffered to 20 mM ammonium bicarbonate using, a sephadex G25 column.

sfGFP-his6 incorporating an unnatural amino acid (BocK or 1) in response to an amber codon at position 145 (psfGFP145TAGPylT-his6) was expressed and purified following the same protocol.

2. Protein Mass Spectrometry

Protein total mass was determined on an LCT time-of-flight mass spectrometer with electrospray ionization (ESI, Micromass). Proteins were rebuffered in 20 mM of ammonium bicarbonate and mixed 1:1 with formic acid (1% in methanol/H2O=1:1). Samples were injected at 10 μl/min and calibration was performed in positive ion mode using horse heart myoglobin. 60 scans were averaged and molecular masses obtained by deconvoluting multiply charged protein mass spectra using MassLynx version 4.1 (Micromass). Theoretical masses of wild-type proteins were calculated using Protparam (http://us.expasy.org/tools/protparam.html), and theoretical masses for unnatural amino acid containing proteins were adjusted manually.

For MS/MS analysis of sfGFP(145-1), the gel band was washed, alkylated, and in-gel digested with trypsin. 1 μl of digest mixture was premixed with 1 μl of CHCA matrix (3 mg/ml in 60% MeCN/0.1% TFA) and 1 μl was applied onto a stainless steel target. The spectrum was acquired with an Ultraflex III TOF/TOF mass spectrometer (Bruker Daltonics, Bremen, Germany). A m/z 2145.972 fragment that matched to peptide modified with 1 was manually selected for further MS/MS fragmentation. The fragmentation ion series confirmed the identity and modification site of the peptide LEYN(1)NSHNVYITADK.

For the analysis of the uncaging process, purified myoglobin was photolysed at 365 nm with a high power LED source module at 365 nm (Black-led-365, Prizmatix). Protein total mass was then determined as described above.

Expression of myo4TAGhis6^{11,12} in the presence of PCKRS/PyltRNA_{CUA} was efficient and dependent on the addition of 1. Electrospray ionization mass spectrometry (ESI-MS) and MS-MS sequencing confirm the incorporation of 1 at a single genetically encoded site (FIG. 3). We confirmed that myoglobin containing 1 is efficiently decaged upon irradiation with 365 nm light in vitro (FIG. 3E).

### Example 4

### Demonstration that the Orthogonal Pair PCKRS/PyltRNA_{CUA} is Functional in Human Cells

To demonstrate that the PCKRS/PyltRNA_{CUA} pair is functional in human embryonic kidney (HEK293) cells, we examined the red and green fluorescence of cells containing mCherry-TAG-egfp-ha (this reporter contains an N-terminal mCherry gene, a linker containing an amber stop codon, a C-terminal enhanced GFP gene, and the HA-tag coding sequence), PCKRS and PyltRNA_{CUA}, in the presence and absence of 1

Protocol

1. Culture, Transfection and Immunoblot Analysis

Adherent human embryonic kidney (HEK)-293 cells were cultured at 37° C. in a 5% CO₂ atmosphere in DMEM+GlutaMAX-1 medium (Gibco) supplemented with 10% FBS and 1× pen-strep solution. Cells were transiently transfected with Genejuice (Novagen) according to the manufacturer's protocol. Double transfections were performed using equal amount of both plasmids. Before transfection, medium was replaced by fresh antibiotic-free medium supplemented, when necessary, with the unnatural amino acid (see figure legends for concentrations). Cells were analyzed 24 h after transfection. For western blot analysis, cells were washed with cold PBS, then lysed with universal lysis buffer (Roche) at 4° C. for 10 min. Western Blots were performed using antibodies against HA-tag (Sigma), Flag-tag (Cell signaling), Ds-Red (Clontech) or p53 (Abcam).

2. Mass Spectrometry Analysis

HEK293 cells in a 100 mm petri dish were transfected with mCherry-TAG-egfp-ha and PCKRS/PyltRNA_{CUA} and grown in presence of 2 mM 1 for 24 h. Cells were lysed and the full length mCherry-1-EGFP-HA was pulled-down using the ProFound™ Mammalian HA Tag IP/Co-IP Kit (Pierce) according to manufacturer's protocol. The protein sample was purified by SDS-PAGE. The protein band of interest was excised from a Coomassie-blue stained gel, washed, alkylated, and in-gel digested with trypsin. A portion of the in-gel digest peptide mixture was separated by nanoscale liquid chromatography (Dionex) on reverse phase C18 column (150×0.075 mm ID, flow rate 0.2 μl/min). The eluate was introduced directly into a LTQ-Orbitrap-XL (Thermo Scientific) mass spectrometer. The spectra were searched against the protein sequence AQASPWH1QLAMVSK (residues 243 to 257 of mCherry-1-EGFP-HA) using in-house MASCOT MS/MS Ions search (www.matrixscience.com). The identity and modification site was confirmed by manual inspection of the fragmentation series.

Microscopy

For imaging cells expressing mCherry-TAG-EGFP-HA, cells were seeded and transfected in 24-well plates. Laser-scanning confocal microscopy was performed using a Bio-Rad Radiance 2100 system mounted on a Nikon Eclipse TE300 inverted microscope equipped with a Plan Fluor ELWD 20×/0.45 objective. Fluorescence emission was measured between 515-530 nm for EGFP (excitation wavelength: 488 nm) and above 560 nm for mCherry (excitation wavelength: 543 nm).

For live cell imaging, cells were seeded and transfected in μ-Dish (Ibidi). Live cells were imaged at room temperature with a Zeiss LSM 710 Laser Scanning Microscope equipped with a Plan Apochromat 63×/1.4 oil immersion objective. Cells were illuminated for 1-5 s (power: 1.2 mW/cm²) with an EXFO X-Cite 120 XL System employing a 120-watt metal halide lamp with a UV filter (filter setting-excitation G 365, beam splitter FT 395, emission BP 445/50), and imaged at room temperature (excitation: 488 nm, emission: 500-560 nm). Microscope settings: for cell images, scan resolution 512×512, averaging 8, scan zoom 3×, scanning speed 10; for real-time imaging, scan resolution 512×512, averaging 1, scan zoom 5× or 3×, scanning speed 8. The mean nuclear (Fn) and cytoplasmic (Fc) fluorescence intensities were quantified using ImageJ software to enable the F(n/c) ratio to be determined according to the formula: F(n/c)=(Fn−Fb)/(Fc−Fb), where Fb is the mean background fluorescence intensity.

Plasmids

The plasmid pCR2.1/htRNA^{Tyr}_{CUA} for expressing human Tyr-tRNA_{CUA} in mammalian cell was a kind gift from Ashton Cropp (University of Maryland). The genes of MbPylRS and MmPylRS, codon optimized for expression in mammalian cells, was purchased from GeneArt.

1. Construction of pMbPylRS-mCherry-TAG-EGFP-HA and pPCKRS-mCherry-TAG-EGFP-HA

We constructed single plasmids that enable the expression of MbPylRS or PCKRS (with an N-terminal Flag-tag) with mCherry-TAG-EGFP (with a C-terminal HA-tag), both under the control of a CMV promoter. To do so, we built a first plasmid pmCherry-TAG-EGFP-HA (allowing the expression of mCherry-TAG-GFP-HA) by generating the EGFP-HA sequence by PCR using pEGFP-N1 (Clonetch) as template and primers mGFPHindamf/AG27, and by then introducing the PCR product in pmCherry-C1 (Clontech) using HindIII and BamHI restriction sites. A multiple cloning site (MCS) was then introduced upstream of the CMV promoter in pmCherry-TAG-EGFP-HA by amplifying the vector backbone with primers 3367bkf/3367bkr, and by then digesting the PCR product with SaclI and religating, giving plasmid pMCS-mCherry-TAG-EGFP-HA. We then amplified by PCR the sequence of Flag-MbPylRS flanked with an upstream CMV promoter and a downstream sequence containing a polyA site with primers KpnpvuKSf/AgesacKSr and using as a template a plasmid initially built by introducing Flag-MbPylRS gene (codon-optimized for mammalian cell expression) into the BamHI and HindIII sites of pcDNA4/TO (Invitrogen). The resulting fragment was then ligated between PvuI and SaclI sites within pMCS-mCherry-TAG-EGFP-HA, giving plasmid pMbPylRS-mCherry-TAG-EGFP-HA. The plasmid pPCKRS-mCherry-TAG-EGFP-HA containing Flag-PCKRS instead of Flag-MbPylRS was generated by cloning Flag-PCKRS gene (codon-optimized for mammalian cell expression) into the AflII and EcoRI sites of pMbPylRS-mCherry-TAG-EGFP-HA. Mutations within PCKRS were introduced by PCR: two fragments were generated using primers AG40/AG43 and AG42/AG41, and MbPylRS gene as template, and then assembled by overlapping PCR using primers AG40/AG41.

2. Construction of p4CMVE-U6-PylT

We constructed a plasmid, p4CMVE-U6-PylT, allowing the expression of PyltRNA_{CUA} in mammalian cells. The expression is driven by a U6 promoter with an upstream CMV enhancer. We first amplified by PCR the CMV enhancer sequence (CMVE) from the CMV promoter of pmCherry-C1 (Clontech) (from 1 to 484) with primers AG16/AG17, digested the PCR product with BamHI and BgIII, and ligated the digested product in pSIREN-Shuttle (Clontech) using BglII site, giving pCMVE-U6. We then generated by PCR a sequence made of PyltRNA_{CUA} DNA sequence, 5′-GGAAACCTGATCATGTAGATCGAATGGACTCTAAATCCGTTCAGCCGGG TTAGATTCCCGGGGTTTCCG-3′, flanked with the 5′-leader 5′-AGATCTTCTAGACTCGAA-3′, and the 3′-trailer 5′-GACAAGTGCGGTTTTT-3′, using primers AG30/AG20 and a plasmid containing PyltRNA_{CUA} sequence as template. The PCR product was then digested with BamHI and MfeI, and ligated in pCMVE-U6 using BamHI and EcoRI sites, giving pCMVE-U6-PylT. Then we generated a cluster of 2 times CMVE-U6-PylT, by cutting the CMV-U6-PylT sequence from pCMVE-U6-PylT with SpeI and EcoRI, and by then ligating the resulting fragment into the NheI and EcoRI sites of pCMVE-U6-PylT, giving p2CMVE-U6-PylT. The plasmid p4CMV-U6-CMV, containing a cluster of 4 times CMVE-U6-PylT, was generated by cutting the cluster of 2 times CMVE-U6-PylT in p2CMVE-U6-PylT with SpeI and EcoRI, and by then ligating the resulting fragment into the NheI and EcoRI sites of p2CMVE-U6-PylT.

Results

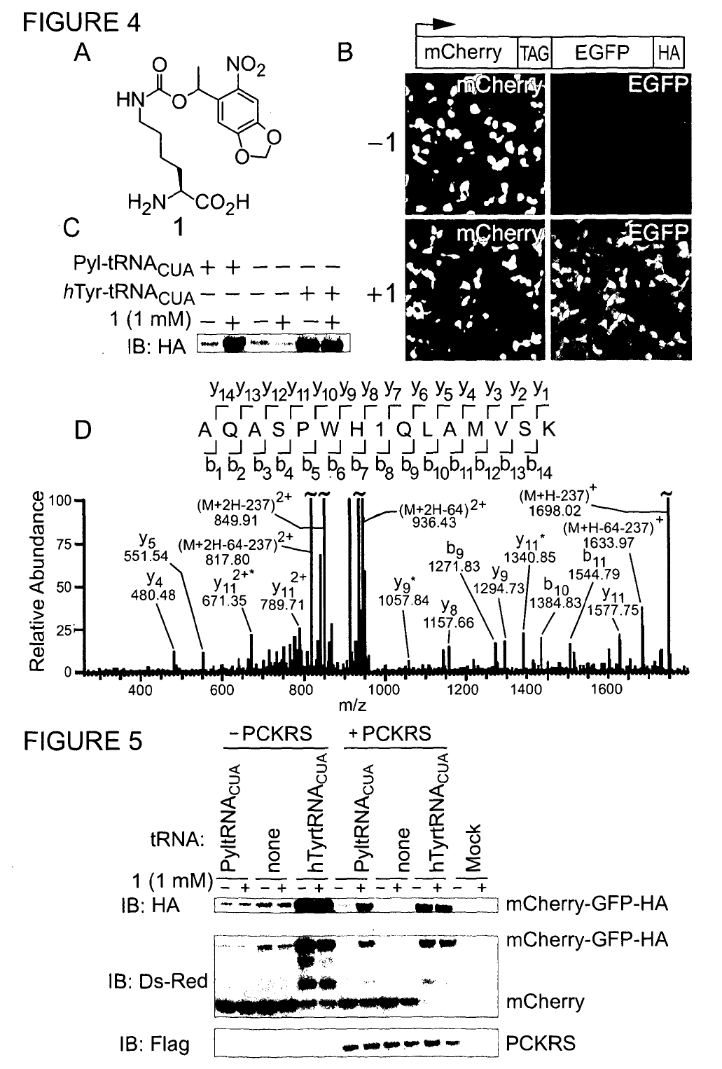

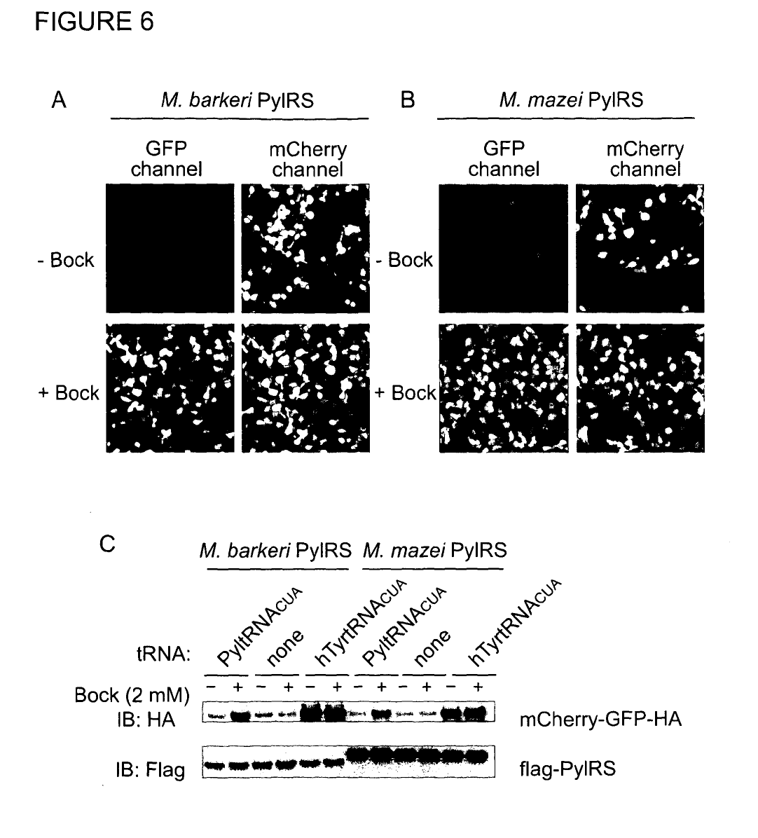

As expected, mCherry fluorescence was detected with or without 1, but EGFP fluorescence was observed only upon addition of 1 (1 mM) (FIG. 4). This confirms that mammalian synthetases do not aminoacylate PyltRNA_{CUA} appreciably in human cells,¹³ and is consistent with the suppression of the amber codon by the PCKRS/PyltRNA_{CUA} pair using 1. Control experiments lacking PyltRNA_{CUA} or PCKRS demonstrate that both are required for amino acid incorporation (FIG. 5). Western blot analysis (FIG. 4C) shows that the efficiency of incorporation of 1 using the PCKRS/PyltRNA_{CUA} pair is comparable to the efficiency of incorporation of tyrosine using a human tyrosyl-tRNA_{CUA} (hTyrtRNA_{CUA}), which is efficiently aminoacylated by the endogenous human tyrosyl-tRNA synthetase. Similar results were obtained with the MbPylRS/PyltRNA_{CUA} pair and E-Boc-protected lysine, a known substrate of PylRS^{13,16} (FIG. 6). The site-specific incorporation of 1 into mCherry-EGFP-HA in mammalian cells was further confirmed by MS/MS sequencing (FIG. 4D).

### Example 5

### Demonstration of the Utility of Photochemical Control by Caged Lysine in Studying Nuclear Import Processes

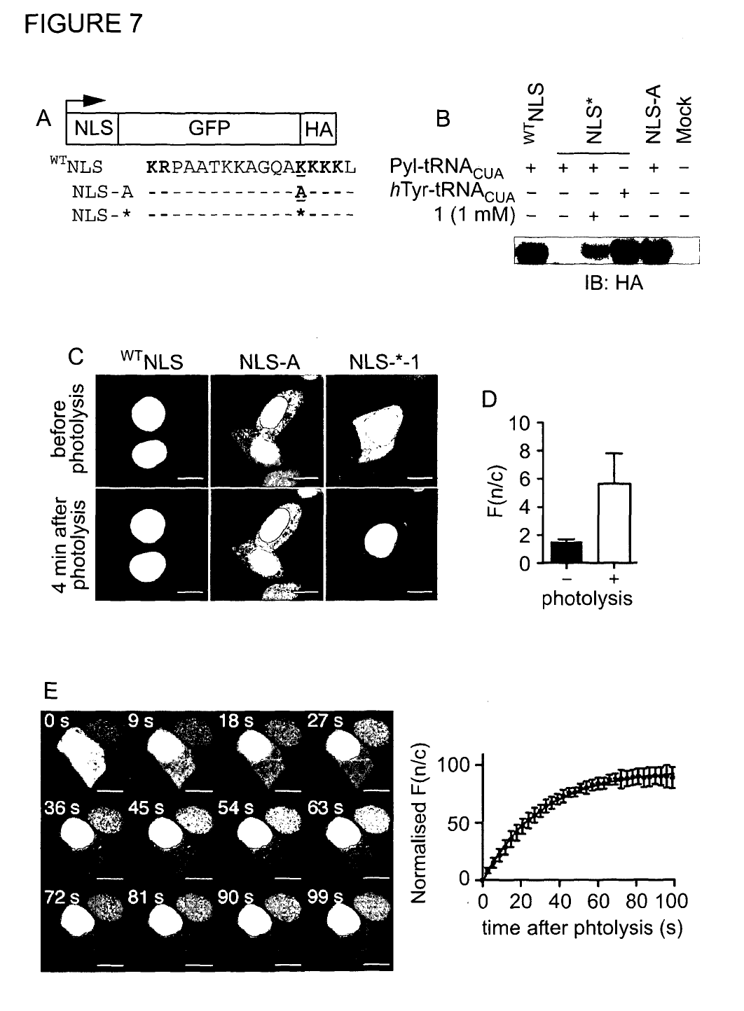

To demonstrate the applicability of 1 for functional studies in mammalian cells, we first investigated its utility for photochemically controlling nuclear import processes. Specifically, we investigated the kinetics of nuclear import driven by the classical bipartite nuclear localization signal (NLS) of nucleoplasmin¹⁷ by caging one of the lysine residues involved in importin-α binding (FIG. 7A). We generated constructs allowing the expression of GFP-HA with an N-terminal wild-type NLS (nls-gfp-ha), with an NLS mutant where the targeted lysine was replaced by an alanine (nls-A-gfp-ha), and with an NLS mutant where the target lysine was replaced by an amber codon (nls-*-gfp-ha).

The protocol were followed as above and the following was also followed:

Construction of pPCKRS-NLS-GFP-HA, pPCKRS-NLS-GCC-GFP-HA, pPCKRS-NLS-TAG-GFP-HA

Plasmids were obtained by ligating PCR fragments for NLS-GFP-HA, NLS-GCC-GFP-HA or NLS-TAG-GFP-HA into the NheI and BsshI sites of pPCKRS-p53-EGFP-HA (see Example 6). As template was used a plasmid provided by Murray Stewart (MRC Laboratory of Molecular Biology, Cambridge UK) containing the nucleoplasmin NLS fused to GFP. The PCR fragment of NLS-GFP-HA was obtained by using primers AG95/AG96. The PCR fragment of NLS-GCC-GFP-HA was obtained by using primers AG95/AG96 to assemble two fragments (generated with primers AG95/AG98 and AG99/AG96) by overlapping PCR. The PCR fragment of NLS-TAG-GFP-HA was obtained by using primers AG95/AG96 to assemble two fragments (generated with primers AG95/AG97 and AG99/AG96) by overlapping PCR.

Results

Expression of full length NLS-GFP-HA protein from nls-*-gfp-ha is dependent on the addition of the PCKRS/PyltRNA_{CUA} pair and 1, demonstrating the incorporation of 1 in response to the amber codon (FIG. 7B). We next confirmed by fluorescence imaging that the photocaged lysine 1 blocks the NLS function as efficiently as the alanine mutation, leading to partial relocalization of the GFP fusions to the cytoplasm (FIG. 7C). GFP is still present in the nucleus because of passive diffusion. Upon photolysis of NLS-*-1-GFP-HA (1 s; 365 nm; 1.2 mW/cm²), we observed nuclear import of cytoplasmic GFP, as a result of the decaging and subsequent nuclear import of GFP (FIG. 7C,D). Quantification of 27 representative cells shows a 3.75-fold increase in the ratio of nuclear to cytoplasmic protein following photolysis (FIG. 7D). Real-time fluorescence microscopy following photolysis allowed us to measure a half-time of ˜20 s for the import of cytosolic GFP (FIG. 7E). Irradiation of cells expressing NLS-A-GFP-HA did not lead to any GFP relocalization (FIG. 7C). Similar results were obtained when suppressing the amber codon in nls-*-gfp-ha with hTyrtRNA_{CUA} (not shown). These results demonstrate that the relocalization is fast and results from specific decaging of 1 upon photolysis.

### Example 6

### Demonstration of the Utility of Photochemical Control by Caged Lysine in Studying More Complicated Nuclear Import Processes Regulated by Numerous Pathways

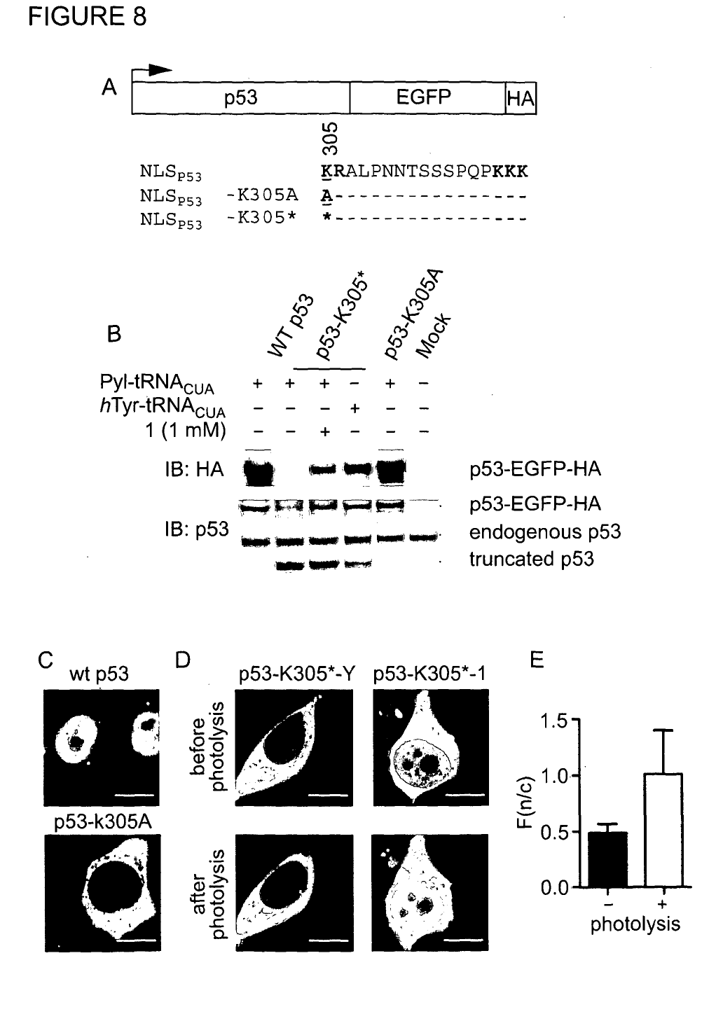

To begin investigating the utility of the photocaging approach in more complicated systems and to investigate the effect of caging one lysine in a system that is regulated by numerous pathways, we next used the photocaged lysine 1 to control the nuclear import of the tumor suppressor p53. p53 nuclear import is carried out by a bipartite nuclear localization signal (NLS) and K305 is a crucial determinant of nuclear localization¹⁸ (FIG. 8A).

We generated constructs allowing the expression of p53 with a C-terminal EGFP-HA tag (p53-egfp-ha) and p53 mutants with either the mutation K305A (p53-K305A-egfp-ha) or an amber codon (p53-K305*-egfp-ha). The same protocols as above were used and the following was also used:

Construction of pPCKRS-p53-EGFP-HA, pPCKRS-p53-305GCC-EGFP-HA, pPCKRS-p53-305TAG-EGFP-HA and pMbPylRS-p53-305TAG-EGFP-HA

Plasmids were obtained by ligating PCR fragments for p53-EGFP-HA, p53-305GCC-EGFP-HA or p53-305TAG-EGFP-HA into the NheI and MfeI sites of pPCKRS-mCherry-TAG-EGFP-HA or pMbPylRS-mCherry-TAG-EGFP-HA. The PCR fragment of p53-EGFP-HA was obtained by using primers AG52/AG55 to assemble two fragments by overlapping PCR: a p53 fragment (generated using primers AG52/AG53, and p53 cDNA as template) and a GFP-HA fragment (generated using primers AG54/AG55, and pmCherry-TAG-EGFP-HA as template). The PCR fragment of p53-K305A-EGFP-HA was obtained by using primers AG52/AG55 to assemble three fragments by overlapping PCR: two fragments from p53 (generated using primers AG52/AG58 and AG56/AG53, and p53 cDNA as template) and the GFP-HA fragment described above. The PCR fragment of p53-305TAG-GFP-HA was obtained by the same strategy using primer AG57 instead of AG58.

Results

The production of full-length p53-EGFP-HA protein from p53-K305*-egfp-ha is dependent on the addition of the PCKRS/PyltRNA_{CUA} pair and 1, confirming the incorporation of 1 in response to the amber codon TAG at position 305 of p53. Western blots (FIG. 8B) demonstrate that the levels of p53 containing 1 were comparable to, but slightly lower than, endogenous p53 levels.

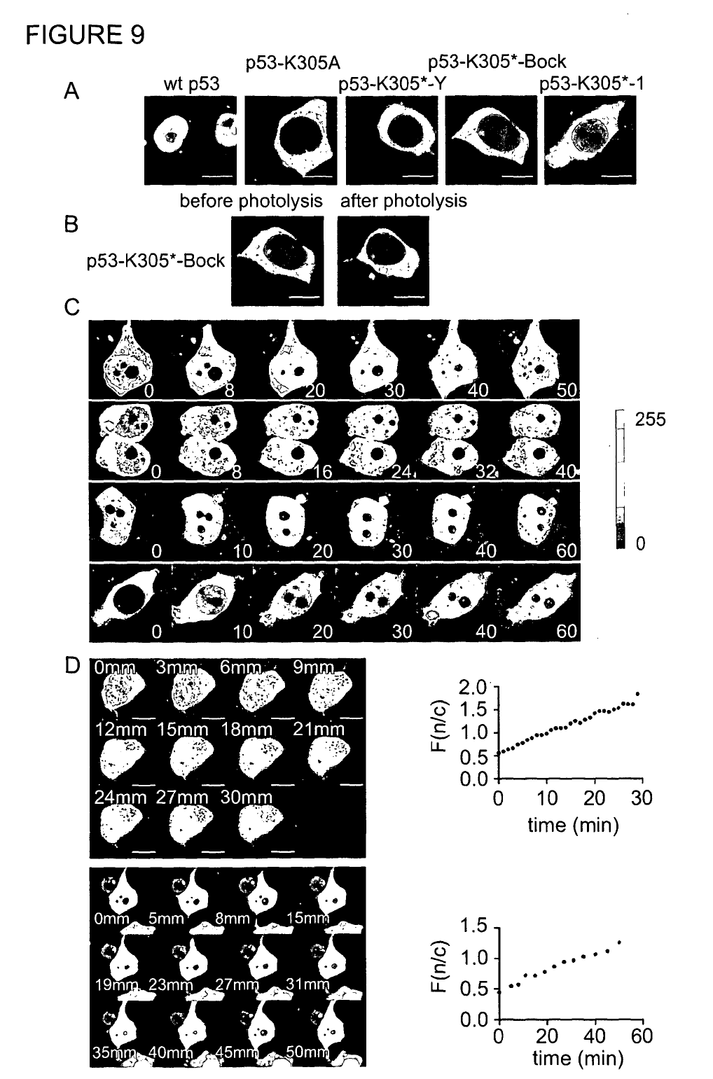

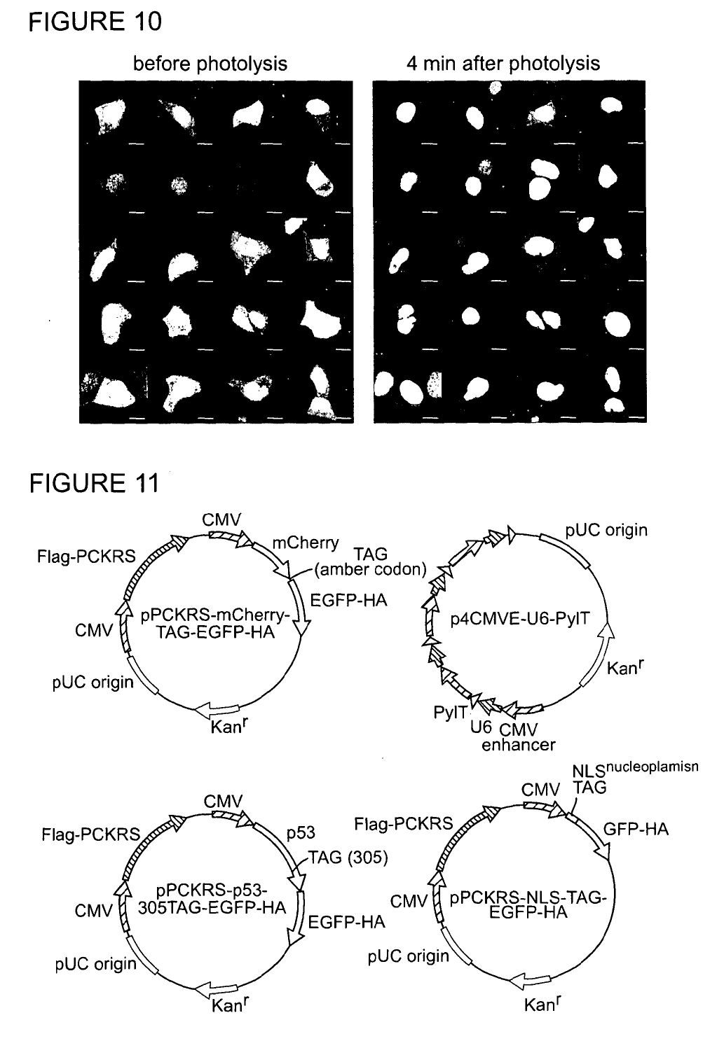

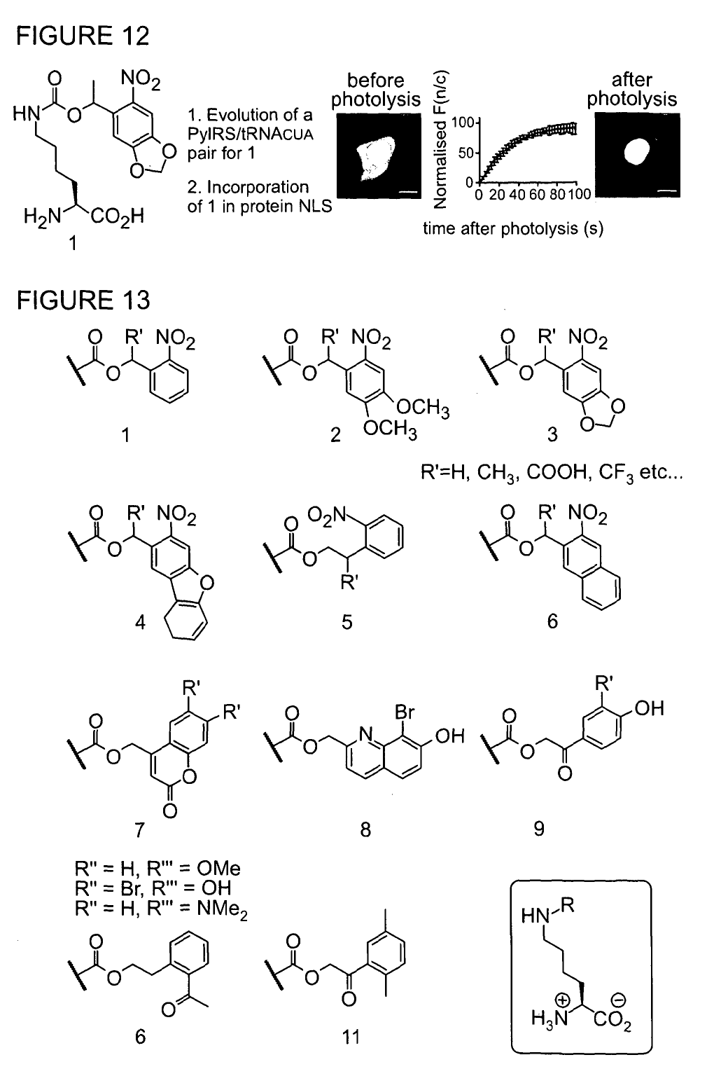

We confirmed by fluorescence imaging that p53-EGFP-HA is localized in the nucleus and that p53-K305A-EGFP-HA is mainly localized in the cytosol, as previously reported¹⁸ (FIG. 8C). When p53-K305*-egfp-ha is expressed together with the PCKRS/PyltRNA_{CUA} pair in presence of 1 (1 mM), we observed that p53 is mainly localized to the cytosol (FIG. 8D and FIG. 9). This demonstrates that the function of the p53 NLS signal has been effectively abrogated through introduction of a single caged lysine. Upon photolysis (5 s; 365 nm; 1.2 mW/cm²) we observe progressive nuclear import of cytoplasmic p53, as a result of the decaging and subsequent nuclear import of p53 (FIG. 8D, FIG. 9). In line with the greater complexity of this system we observe greater cell-to-cell variability in nuclear import than in the nucleoplasmin case. In control experiments we incorporated ε-Boc-protected lysine or tyrosine in response to the amber codon at position 305 of p53. These p53 variants were localized to the cytoplasm and did not localize to the nucleus following photolysis (FIG. 8D, FIG. 9), confirming that the re-localization results from the specific decaging of 1.

In conclusion we have demonstrated the synthesis and site-specific genetic incorporation of the new photocaged lysine 1 into proteins in human cells. We have used this amino acid to cage nuclear localization signals and measure the kinetics of nuclear import via the photochemical control of protein localization in human cells using a rapid pulse of non-photodamaging UV irradiation.

### Example 7

### Engineered Light-Activated Kinases Enable Temporal Dissection of Signalling Networks in Living Cells

The activation of user-defined kinases with high temporal resolution inside living cells would accelerate our understanding of signal transduction. We report a general strategy for creating light-activated kinases. Photo-activatable MEK1 allows the specific, rapid, and receptor independent activation of a sub-network within MAP kinase signalling. Time-lapse microscopy allowed us to observe ERK2 translocation following MEK1 photo-activation with high temporal resolution in single mammalian cells. The photo-activated sub-network exhibits much less cell-to-cell variability than the EGF stimulated pathway. While ERK2 nuclear levels rise upon exposure to EGF, before returning to pre-stimulus levels, the photo-activated sub-network results in sustained levels of nuclear ERK2. The MAP kinase pathway upstream of MEK1 introduces a delay prior to ERK2 translocation, but does not limit the kinetics of translocation once initiated. ERK2 accumulation in the nucleus following MEK1 photo-activation exhibits a sigmoidal time course, consistent with non-processive (distributive), dual-phosphorylation of ERK2 by MEK1 being rate-determining for nuclear import.

### Introduction

Organisms survive, develop and respond to environmental changes by temporally and spatially regulating complex signalling networks. Understanding the dynamic processes by which signalling networks transmit information in normal physiology and in disease is an important goal.

Protein kinases are arguably the most important class of signalling proteins. This large class of enzymes (containing more than 500 members (Manning et al., 2002)) transfer the gamma phosphate from ATP to specific tyrosine, threonine or serine residues on a target protein. Almost every biological process is regulated by phosphorylation—including metabolic processes, cell-cycle progression, cytoskeletal rearrangement, organelle trafficking, membrane transport, muscle contraction, growth, apoptosis and differentiation, immunity and learning and memory (Manning et al., 2002)

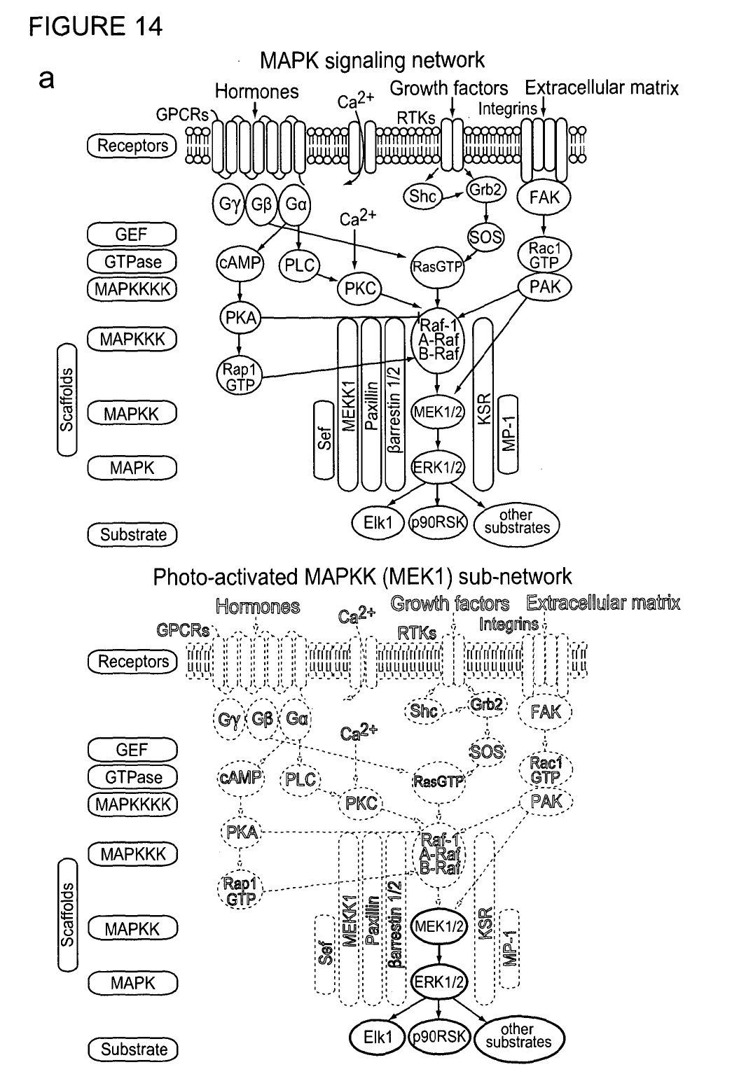

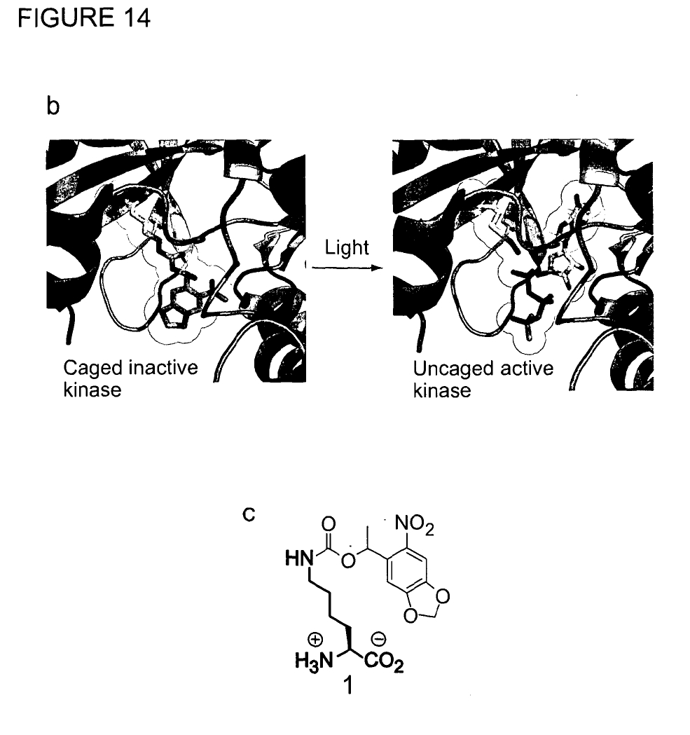

As our understanding of connectivity in kinase mediated networks expands (Breitkreutz et al.) it is becoming increasingly clear that signal transduction pathways are complex, dynamic, multistep processes that may crosstalk, feed-back, feed-forward and contain elementary steps that operate at very different rates. The complexity of signalling networks make it difficult to assign the molecular cause and effect for events between a pathway's extracellular inputs and its outputs. We realized that the ability to rapidly and specifically target the activation of a single kinase within the cell should make it possible to dissect the cell's signalling network into simpler sub-networks (FIG. 14a). These simpler sub-networks may be more amenable to study, and may allow us to directly observe the kinetics of events that are un-resolved within the context of an entire network.

Several methods have been reported to control the activity of protein kinases, including induced dimerization (Spencer et al., 1993), controlled degradation (Banaszynski et al., 2006), engineered allosteric activation (Karginov et al., 2010), chemical rescue of an inactivating mutation (Qiao et al., 2006) and selective inhibition of sensitized kinases (Bishop et al., 2000). While these methods have contributed substantially to our knowledge of kinase function (Burkard et al., 2007; Choi et al., 2008; Justman et al., 2009; Kim et al., 2008; Larochelle et al., 2006; Li et al., 2009; Ventura et al., 2006), they a) may be limited to specific kinases, b) inactivate rather than activate kinases, c) may not allow regulation of kinase catalytic activity independent of other roles the kinase may play—such as acting as a scaffold or an anchor, and d) do not allow the study of rapid processes that occur within seconds following activation of a kinase's catalytic activity.

A potentially attractive strategy for rapidly activating protein function inside living cells involves replacing a key amino acid in the protein with a photo-caged version of the amino acid, leading to an inactive protein. Upon illumination of the protein, the photo-cage is removed and the native function of the protein is restored (Deiters; Deiters, 2009; Lawrence, 2005; Lee et al., 2009). Chemical and enzymatic methods including native chemical ligation and in vitro translation have been used to introduce photo-caging groups into proteins in vitro (Endo et al., 2004; Ghosh et al., 2004; Pellois et al., 2004). These approaches have been extended for the introduction of caged proteins into eukaryotic cells by permealization (Hahn and Muir, 2004) or microinjection (Pellois and Muir, 2005), but these methods remain challenging. In one case a serine phosphorylation site in S. cerevisiae was masked using a photocaged serine installed into Pho4 using an evolved leucyl-tRNA synthetase/tRNA_{cu}A pair that incorporates a photo-caged serine in response to the amber codon in yeast (Lemke et al., 2007), but this approach—which regulates substrate availability rather than kinase activity—has not been demonstrated in mammalian cells. Moreover, this approach cannot be used to study tyrosine phosphorylation, or the majority of processes that are regulated by multiple phosphorylations, including combinations of tyrosine phosphorylation, threonine phosphorylation and serine phosphorylation.

We recently reported an evolved variant of the M. barkeri pyrrolysyl-tRNA synthetase/tRNA_{CUA}, the photocaged lysyl-tRNA synthetase/RNA_{CUA} (PCKRS/tRNA_{CUA}) pair, that directs the incorporation of the photocaged amino acid 1 (FIG. 14c) into proteins in mammalian cells in response to the amber stop codon (Gautier et al., 2010). We incorporated this amino acid into nuclear localization sequences of proteins, blocking their nuclear import function, and mis-localizing the proteins to the cytosol. Upon decaging with a one-second pulse of light, 1 was converted to lysine on the protein—restoring its nuclear localization sequence—and we were able to follow the kinetics of nuclear localization in real time (Gautier et al., 2010).

Since many protein kinases can be constitutively activated by deletions in their regulatory domains or by mutations of their phosphorylation (activation) sites to negatively charged amino acids (Cowley et al., 1994; Huang et al., 1997; Mansour et al., 1994; Minden et al., 1994; Raingeaud et al., 1995), we realized that it may be possible to create a kinase that is poised for photo-activation by simultaneously introducing activating mutations into the kinase and photo-caging a key residue in the active site of the enzyme.

Protein kinases contain a near universally conserved lysine residue in their ATP binding pocket that anchors and orientates ATP (Manning et al., 2002). Modelling 1 in place of the conserved lysine in several kinase active sites revealed that the bulky caging group should prevent ATP binding but may be accommodated within the active site without perturbing the kinase structure (FIG. 14b).

Here we report a general strategy for creating kinases that can be activated with light, and apply our approach to provide new insight into a conserved MAP kinase pathway, which is important in cell proliferation, survival, differentiation, apoptosis, motility and metabolism. We create a version of MEK1 kinase, that can be photo-activated with a 1-2 second pulse of light, allowing the specific, rapid, and receptor independent activation of a sub-network in which MEK1 phosphorylates ERK1/2 on both a threonine and a tyrosine residue, leading to ERK1/2 accumulation in the nucleus and the phosphorylation and activation of transcription factors important for neural differentiation in PC12 cells and cell cycle re-entry and initiation of DNA synthesis in fibroblasts (Dikic et al., 1994; Lenormand et al., 1993; Traverse et al., 1994).

Time-lapse microscopy allowed us to follow ERK2 nuclear accumulation with high temporal resolution following MEK1 photo-activation in single cells. These experiments revealed that the photo-activated sub-network exhibits much less cell-to-cell variability than the EGF stimulated pathway. While EGF stimulation results in exact adaptation (Cohen-Saidon et al., 2009) (a phenomenon in which ERK2 nuclear levels rise upon exposure to EGF, but then return to pre-stimulus levels), the pool of photo-actived MEK1 acts as a stationary stimulus that maintains high levels of ERK2 in the nucleus for long periods of time. Our results reveal that the MAP kinase pathway upstream of MEK1 introduces a delay prior to ERK2 translocation, but does not limit the kinetics of translocation once initiated. The accumulation of ERK2 in the nucleus following photo-activation of MEK1 is sigmoidal with time, consistent with the non-processive (distributive) (Burack and Sturgill, 1997; Salazar and Hofer, 2009), dual-phosphorylation of ERK2 by MEK1 being rate-determining for nuclear import.

### Results

### Controlling a MAP Kinase Sub-Network Via MEK-1 Photo-Activation

To create a MEK1 mutant that could be activated upon illumination, we first constructed a constitutively active MEK1 mutant, A-MEK1-ΔN (A denotes active), in which residues 30-49 are deleted (Mansour et al., 1994). We replaced lysine K97, a near-universally conserved lysine crucial for ATP binding and catalysis, by the photocaged lysine 1 in A-MEK1-ΔN by replacing the codon for K97 with an amber stop codon—creating mek1-ΔN-97TAG—and directed the incorporation of 1 in response to this codon using the evolved PCKRS/tRNA_{CUA} pair (Gautier et al., 2010). This produced C-MEK1-ΔN, in which C denotes that the catalytic residue K97 is caged by genetically incorporating amino acid 1.

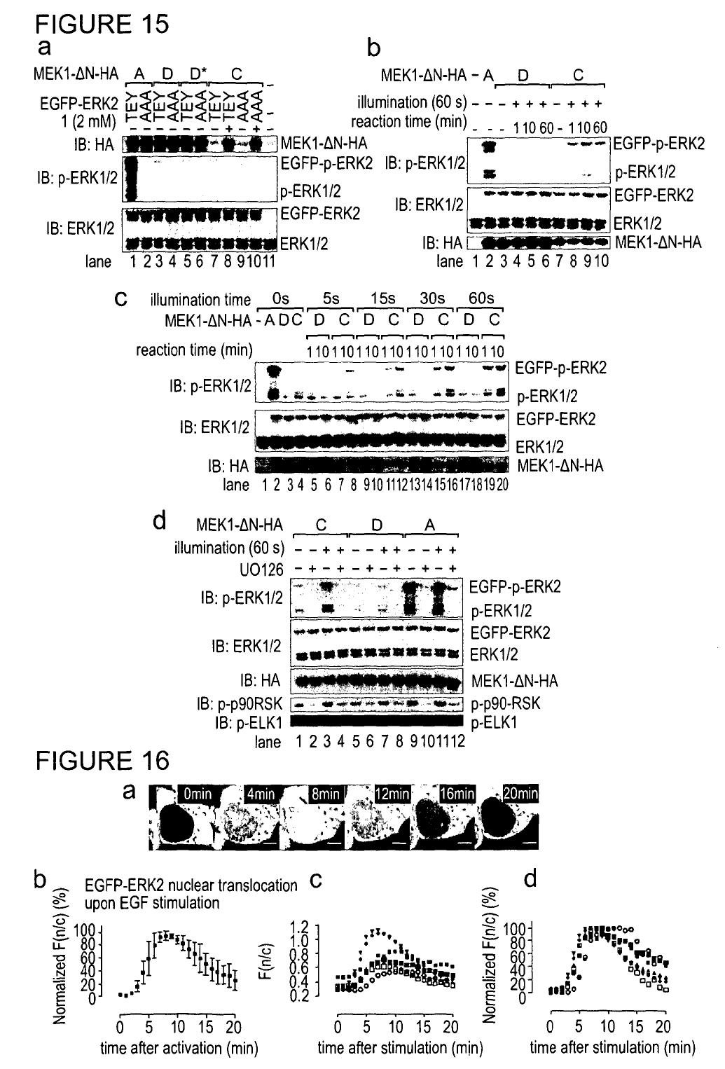

Immunoblotting showed that the level of C-MEK1-ΔN protein in human embryonic kidney (HEK) 293T cells transfected with mek1-ΔN-97TAG and expressing the PCKRS/tRNA_{CUA} pair when grown in the presence of 1 (2 mM) was comparable with that obtained when tyrosine was incorporated instead of 1 using a human tyrosine amber suppressor tRNA^{Tyr}_{CUA} (FIG. 15a). Similarly, the level of C-MEK1-ΔN was comparable to that of A-MEK1-ΔN and the D-MEK1-ΔN mutant (D denotes dead), which contains a known mutation, K97M, that abolishes kinase catalytic activity (FIG. 15a and FIG. 21). Taken together these observations demonstrate that the photo-caged kinase can be produced at levels comparable to the wild-type kinase, and that the levels of these proteins in transfected cells are comparable to those of endogenous MEK1 (FIG. 21b), which is present in cells at micromolar concentrations.

MEK1 is highly specific for the downstream extracellular signal-regulated protein kinases ERK1 and ERK2, and has no other known substrates (Shaul and Seger, 2007). MEK1 phosphorylates two regulatory residues in ERK1/2, a threonine and a tyrosine, both part of a conserved Thr-Glu-Tyr (TEY) motif (Payne et al., 1991). To verify that the introduction of the photocaged lysine 1 prevents kinase activity, C-MEK1-ΔN was co-expressed with ERK2 fused to an enhanced green fluorescence protein (EGFP-ERK2) in resting HEK293ET cells. Immunoblotting revealed no phosphorylation of the TEY motif in both endogenous ERK1/2 and EGFP-ERK2 (FIG. 15a), showing that the caged lysine 1 blocks the catalytic activity of C-MEK1-ΔN as efficiently as the K97M mutation in D-MEK1-ΔN.

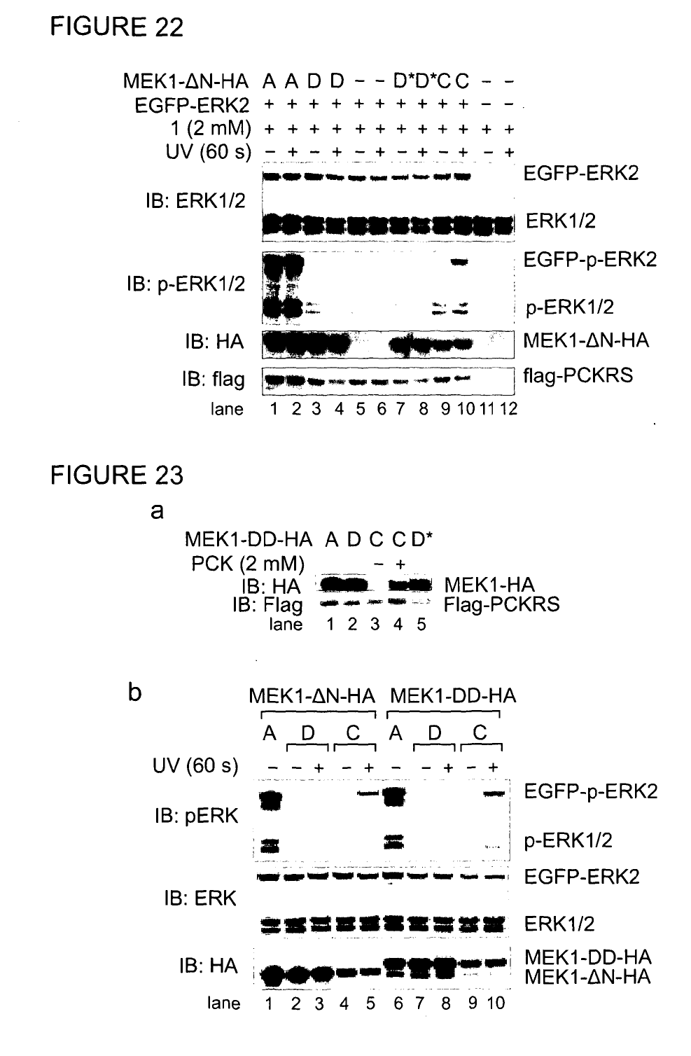

To activate C-MEK1-ΔN we illuminated cells producing the protein for one minute using a 365 nm light-emitting diode (LED) lamp placed underneath the culture plate. This method allows us to illuminate a sufficient number of cells for western blot analysis with monochromatic light that avoids sample heating. Immunoblotting revealed phosphorylation of EGFP-ERK2 and endogenous ERK2 one minute after illumination of cells, with a maximum phosphorylation around 10 minutes after illumination (FIG. 15b and FIG. 22). We observed more phosphorylated EGFP-ERK2 than phosphorylated endogenous ERK1/2. This is consistent with the fact that only a subset of cells contain EGFP-ERK2 and C-MEK1-ΔN, via transfection, while essentially all cells contain endogenous ERK1/2. These observations suggest that phosphorylation directly results from uncaged co-expressed C-MEK1-ΔN rather than by an illumination-induced cellular stress response that would affect all cells and activate the endogenous MAP kinase pathway leading to phosphorylation of ERK. Control experiments replacing C-MEK1-ΔN with the inactive kinase D-MEK1-ΔN (FIG. 15b and FIG. 22) or with no protein (FIG. 22) led to no detectable phosphorylation of EGFP-ERK2 or endogenous ERK1/2 after illumination. These control experiments further demonstrate that illumination does not activate the endogenous MAP kinase pathway and lead to phosphorylation of ERK. EGFP-ERK2 levels in transfected cells are lower-than or equal to that of the endogenous ERKs.

Illumination of cells co-expressing C-MEK1-ΔN and EGFP-ERK2 for increasing times led to increased phosphorylation of EGFP-ERK2 (FIG. 15c), demonstrating that it is possible to control, with high precision, the cellular concentration of active kinase by simply adjusting the illumination time.

To demonstrate that photo-activation of C-MEK1-ΔN led to phosphorylation of ERK1/2 substrates we probed the phosphorylation state of p90 ribosomal S6 kinase p90RSK and the transcription factor Elk-1, two downstream substrates of ERK1/2. Illumination of resting cells co-expressing C-MEK1-ΔN and EGFP-ERK2 led to an increase of endogenous phosphorylated p90RSK and Elk-1, which was not observed when D-MEK1-ΔN was used instead of C-MEK1-ΔN or when the MEK1 inhibitor U0126 was added (FIG. 15d). These data demonstrate that photo-activation of C-MEK1-ΔN allows us to specifically activate a sub-network of the MAP kinase pathway in the cell, in which ERK1/2 is phosphorylated and subsequently phosphorylates p90RSK and Elk-1.

EGF Stimulation Leads to ERK Translocation Following a Long Lag-Phase with High Cell-to-Cell Variability

Specifically activating a sub-network within the entire cell might allow us to observe the kinetics of processes in the sub-network directly that would be difficult to observe when the whole pathway is activated. Aside from its role as an activator, MEK1 also acts as a cytoplasmic anchor protein for ERK1/2 (Fukuda et al., 1997; Rubinfeld et al., 1999). Upon dual phosphorylation, ERK1/2 detaches from MEK1 and its other cytoplasmic anchors and translocates into the nucleus (Khokhlatchev et al., 1998; Rubinfeld et al., 1999), where it regulates gene expression by phosphorylating transcription factors (Brunet et al., 1999; Chen et al., 1992; Kim et al., 2000; Lenormand et al., 1993). Dephosphorylation of nuclear ERK1/2 returns it to the nucleus (Ando et al., 2004; Costa et al., 2006; Volmat et al., 2001). In vitro, it is known that MEK1 mediated phosphorylation of ERK1/2 on its two phosphorylation sites is non-processive (distributive), and distributive phosphorylation of ERK1/2 leads to ultrasensitive, switch-like, sigmoidal kinetics for formation of the di-phosphorylated form (Burack and Sturgill, 1997; Ferrell and Bhatt, 1997; Markevich et al., 2004; Salazar and Hofer, 2009). However in vivo, where scaffolds and other proteins may organize and regulate kinases (Bashor et al., 2008; Malleshaiah et al.), it is unknown if sigmoidal kinetics for this elementary step are conserved, and whether these phosphorylations, or steps upstream of MEK, control ERK1/2 translocation (Lidke et al., 2010). We set out to compare the kinetics of a) ERK1/2 translocation following receptor mediated stimulation of the whole pathway to the kinetics of b) ERK translocation in the photoactivated sub-network, with the goal of providing insight into how this pathway controls the kinetics of ERK1/2 translocation, a key regulatory step in controlling ERK responsive transcription factors.

When cells expressing wild-type MEK1 and EGFP-ERK2 are stimulated with 100 ng/ml of epidermal growth factor (EGF) the entire MAP kinase pathway is activated and EGFP-ERK2 is translocated to the nucleus. Quantification of fluorescence time-lapse microscopy images demonstrates that the nuclear accumulation of EGFP-ERK2 exhibits sigmoidal kinetics, with a lag phase of 3 minutes prior to rapid nuclear accumulation of EGFP-ERK2 (FIG. 16a,b). We observe substantial cell-to-cell variability in both the t_{1/2} for import and the maximal ratio of nuclear to cytoplasmic EGFP-ERK2, and, as previously reported (Cohen-Saidon et al., 2009), we observe less variability in the timing of nuclear import than in the fraction of ERK2 in the nucleus following stimulation (Cohen-Saidon et al., 2009) (FIG. 16c,d). The nuclear accumulation of EGFP-ERK2 peaks and then dissipates to pre-stimulus levels, a well known effect in cellular systems known as exact adaptation (Cohen-Saidon et al., 2009). This effect is not understood in molecular detail for EGF signalling, but must involve cellular processes that compensate for EGF stimulation at some—as yet undefined—point or points in the pathway. Our experiments using transfected cells reproduce the previous observations on cell-to-cell variability following EGF stimulation using stable cell lines with endogenous MEK1 and a fluorescently tagged MEK2 produced from the endogenous promoter at endogenous levels (Cohen-Saidon et al., 2009). This further confirms that our experiments reflect the endogenous situation.

C-MEK1 Photo-Activation Leads to Rapid ERK Translocation, which is Highly Reproducible from Cell to Cell

To characterize the kinetics of ERK2 nuclear translocation upon light-activation of MEK1, we required a new photo-activatable MEK1. C-MEK1-ΔN could not be used to sequester ERK2 in the cytoplasm because the N-terminal sequence (residues 30-49) deleted for rendering MEK1 constitutively active also contains the nuclear export sequence (NES, residues 33-44) responsible for MEK1 cytoplasmic localization (Fukuda et al., 1996). We created a new caged MEK1 with an N-terminal NES by photo-caging the conserved lysine K97 of a constitutively active MEK1 in which Ser218 and Ser222, that are normally phosphorylated by Raf to activate MEK1, are substituted with Asp residues (A-MEK1-DD), mimicking the phosphorylated state (Mansour et al., 1994). We confirmed by immunoblotting experiments that the new photoactivatable caged MEK1 (C-MEK1-DD) functions as efficiently as C-MEK1-ΔN (FIG. 23).

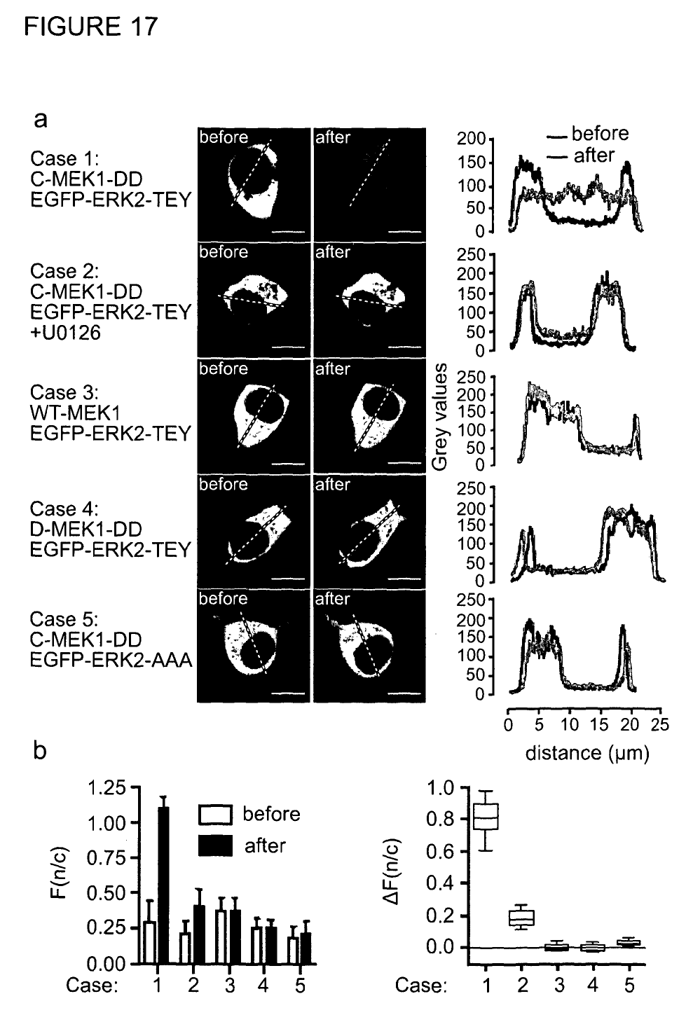

Confocal fluorescence imaging showed that C-MEK1-DD retains EGFP-ERK2 in the cytoplasm, demonstrating that its anchoring function is maintained, while its catalytic activity is eliminated (FIG. 17a). Upon illumination with a microscope metal halide lamp equipped with a UV filter (2 s, 365 nm, 1 mW/cm²), cells under resting conditions that contain cytoplasmic C-MEK1-DD and EGFP-ERK2 rapidly accumulated EGFP-ERK2 in the nucleus (FIG. 17a,b). A 4-fold increase in the ratio of the nuclear to cytoplasmic EGFP fluorescence F(n/c) within ten minutes was observed (FIG. 17b). Adding 10 μM of MEK1 inhibitor U0126 before illumination significantly blocked nuclear accumulation (FIG. 17a,b). Likewise, when resting cells co-expressing EGFP-ERK2 with wild-type wt-MEK1 or with the catalytically dead mutant D-MEK1-DD were illuminated, no nuclear translocation was observed (FIG. 17a,b). These experiments show that EGFP-ERK2 is translocated in resting cells upon phosphorylation by light-activated C-MEK1-DD. Furthermore, when C-MEK1-DD was co-expressed with an EGFP-ERK2 mutant in which the phosphorylation site (TEY) was mutated to AAA (EGFP-ERK2-AAA), no translocation of EGFP-ERK2-AAA was observed upon illumination (FIG. 17a,b), in accordance with nuclear translocation driven by dual phosphorylation of the TEY motif. These data show that activation of ERK2 by MEK1-induced phosphorylation of the TEY motif is sufficient for triggering ERK2 nuclear translocation.

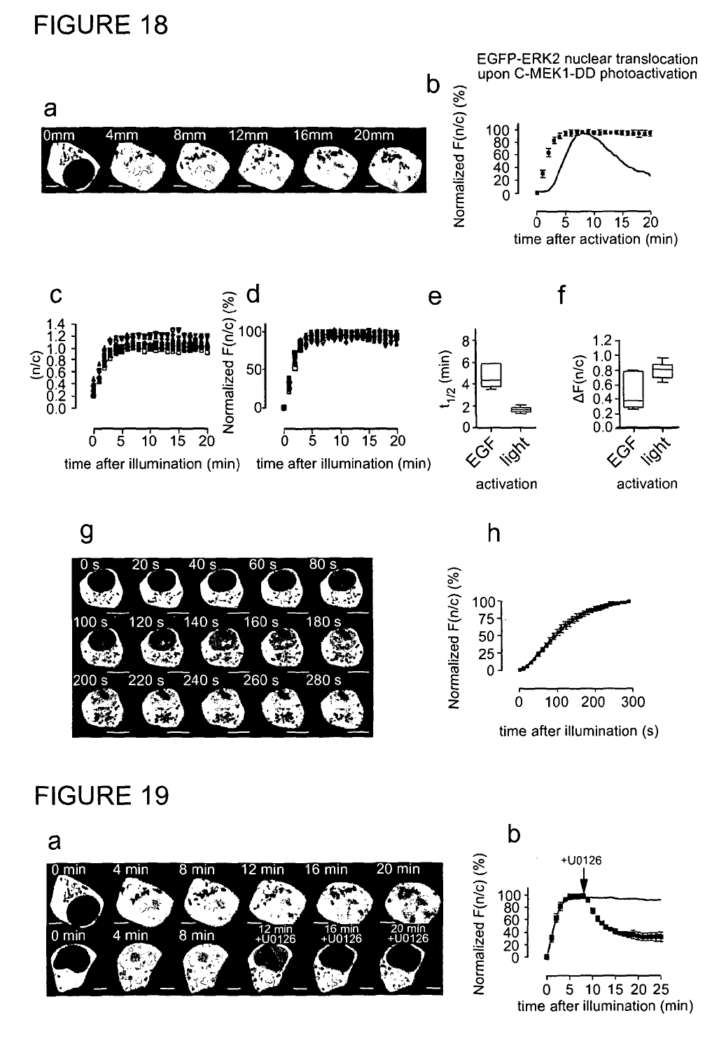

Real time measurements of EGFP-ERK2 nuclear translocation show that the translocation process in the sub-network is initiated much faster after photoactivation of C-MEK1-DD than for EGF-stimulated activation of the whole pathway (t_{1/2}=1.5 min vs 4.5 min, FIG. 18b).

Photoactivation of C-MEK1-DD in contrast to EGF stimulation, allows the nuclear accumulation of EGFP-ERK2 to be sustained for long periods (FIG. 18a,b, FIG. 16b). This shows that the pool of photo-activated C-MEK1-DD acts as a stationary stimulus, and that, unlike EGF stimulation of the whole pathway, the activity of the sub-network is not subject to exact adaptation. These observations suggest that the molecular targets that are adaptively regulated in the EGF response, and the regulators that act on these targets, cannot both be located between MEK1 and ERK2 in the MAPK network.

We observed much less cell-to-cell variability for the rate of translocation and the increase of nuclear fluorescence when photo-activating C-MEK1-DD than when stimulating wild-type MEK1 with EGF (FIG. 18c-f), showing that the photo-activated sub-network is more less sensitive to cell-to-cell variability than the whole network activated by the external stimuli. The robustness of such sub-networks should aid quantitative measurement.

The stationary stimulus for nuclear import of ERK1/2 is counteracted by nuclear dephosphorylation of ERK1/2 and its export (Ando et al., 2004; Costa et al., 2006; Volmat et al., 2001). To unveil this compensating process and visualize ERK2 nuclear export, we induced EGFP-ERK2 nuclear translocation by C-MEK1-DD photoactivation, and then blocked MEK1-induced ERK2 nuclear translocation by addition of the MEK1 inhibitor U0126. This caused a rapid loss of EGFP nuclear fluorescence (t_{1/2}<3 min) (FIG. 19a,b, Supplementary movie S1), in accordance with a rapid dephosphorylation-induced nuclear export of the imported ERK2 (Ando et al., 2004; Costa et al., 2006). These data demonstrate that action of U0126 on MEK1 is sufficient to account for its effects on the MAP kinase pathway.

### The Kinetics of ERK Translocation are Regulated by MEK1 Mediated Phosphorylation

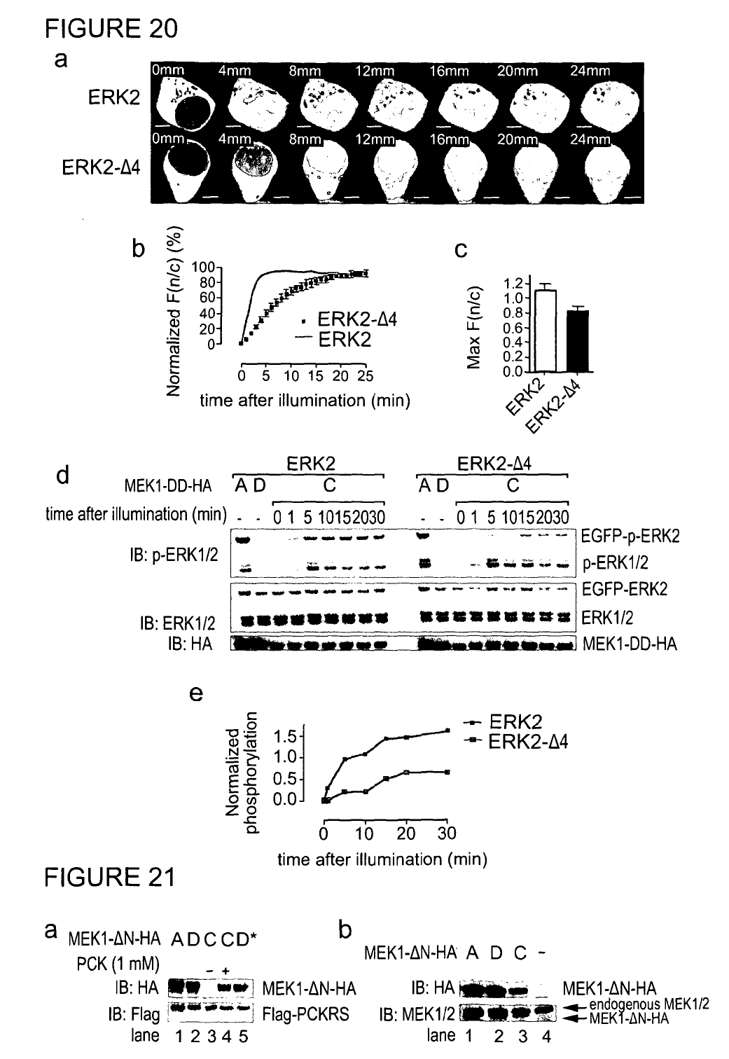

Fluorescence time-lapse microscopy, with high temporal resolution, reveals a sigmoidal curve for EGFP-ERK2 translocation. The sigmoidal curves for EGF stimulated translocation (FIG. 16b) and photo-activated translocation (FIG. 18g,h and Supplementary movie S2) have comparable slopes once translocation is initiated (as judged by the slope when 50% of the net translocation has occurred), but the initial lag phase in the EGF stimulated experiment is much longer than in the photo-activated experiment (Compare FIG. 18g,h and FIG. 16b). These observations suggest that steps upstream of MEK1 in the pathway function to introduce a delay prior to activating the translocation process, but do not significantly affect the translocation rate once translocation has begun. The rate of translocation is therefore set between MEK1 and ERK2 in the pathway. The sigmoidal curve for the sub-network (FIG. 18h), is consistent with the distributive dual phosphorylation of ERK1/2 by MEK1, previously only observed in vitro, operating in vivo to determine the rate of translocation in cells (Ferrell and Bhatt, 1997; Salazar and Hofer, 2009). We followed the accumulation of phosphorylated ERK1/2 by western blot following activation of the sub-network with an LED lamp for 1 minute. Phosphorylated ERK1/2 accumulated rapidly, but the time-resolution of this method does not allow us to directly observe the lag-phase in the formation of the doubly phosphorylated species (FIG. 20 d,e).

To further characterize the net kinetics of EGFP-ERK2 translocation and their relationship with the rate of EGFP-ERK2 phosphorylation, we studied the rate of translocation of the ERK2-Δ4 mutant that contains the deletion Δ174-177 and that has been reported to have an altered nuclear import rate, but an unchanged export rate (Lidke et al., 2010). When we photoactivated cells co-expressing this mutant with C-MEK1-DD we observed nuclear translocation, but with slower kinetics (t_{1/2}=6 min) (FIG. 20a,b), accompanied by a lower level of nuclear accumulation (FIG. 20c). The slower nuclear accumulation suggests that the rate of nuclear import is now only slightly faster than the rate of export. Both the lag time following photoactivation and the net rate of import (as judged by the slope when 50% of the net translocation has occurred) have changed from the wild-type case. The coupled change in lag time and net rate of import is consistent with the distributive dual phosphorylation of ERK1/2 being the rate-limiting step in its nuclear import in the wild-type case. Our data suggest that the slower translocation for this mutant may be due to slower phosphorylation, as has been recently proposed (Lidke et al., 2010). If we consider that the rates of import and export are driven by the rates of the respective phosphorylation and dephosphorylation, the slower nuclear import and diminished nuclear accumulation observed for EGFP-ERK2-Δ4 should be accompanied by a slower and diminished accumulation of phosphorylated EGFP-ERK2-M. Immunoblotting of cells co-expressing EGFP-ERK2-Δ4 and C-MEK1-DD and illuminated with a LED lamp for 1 minute revealed that the apparent rate of phosphorylation of EGFP-ERK2-Δ4 is slower and that the degree of phosphorylation of EGFP-ERK2-Δ4 is less than that of EGFP-ERK2 (FIG. 20d-e). This experiment directly correlates the slower and diminished nuclear accumulation of EGFP-ERK2-Δ4 with a slower phosphorylation, providing further evidence that nuclear translocation of ERK1/2 is controlled by the rate of double phosphorylation by MEK1.

### Discussion

In conclusion, we have demonstrated a strategy for creating kinases that can be activated in living mammalian cells by a 1-2 second light pulse. We have shown that MEK1 catalytic activity can be inactivated by genetically encoding the photocaged lysine 1, while maintaining its anchoring function. We have demonstrated that the amount of activated MEK1 can be controlled by the duration of illumination and that our method allows us to turn-on a sub-network within MAP kinase signalling independent of the activation of the pathway through extracellular receptors.

We have demonstrated that the sub-network activation kinetics show less cell-to-cell variability upon photoactivation than EGF mediated activation kinetics. This observation may reflect an evolutionarily conserved role for the MEK1 sub-network in reproducibly transmitting signals resulting from diverse extracellular inputs, without distorting these signals by introducing additional noise. Such a model might allow the transcriptional program initiated upon ERK1/2 import to accurately respond to the level and intensity of extracellular stimulation.

Unlike the EGF activated pathway the sub-network is not controlled by exact adaptation. This suggests that cell-to-cell variation may result from processes upstream of MEK activation and that the pathway downstream of MEK is not sufficient to elicit the adaptive response in ERK2 translocation elicited by EGF stimulation.

By comparing EGF activation and sub-network activation, we conclude that steps prior to MEK1 create a delay of several minutes in initiating ERK1/2 translocation following EGF stimulation—leading to switch-like activation of MEK1, but do not dramatically alter the transport kinetics of ERK1/2 once transport is initiated, revealing that steps prior to MEK1 are not rate determining for ERK1/2 translocation. Finally, we show that the rate of ERK1/2 translocation following MEK1 photo-activation displays a lag phase, and that mutations that affect the phosphorylation of ERK1/2 directly also directly affect the kinetics of ERK1/2 nuclear accumulation following MEK1 activation. These results suggest that the distributive dual phosphorylation of ERK1/2 by MEK1 is rate determining for ERK transport in this MAP kinase pathway.

The methodology presented here can be used to provide very high temporal and spatial resolution (Levskaya et al., 2009) to address the effects of spatial and temporal kinase activation in cells. Due to the substantial conservation of the targeted lysine residue, the light-activation method reported here should be generally and readily applicable to creating photoactivated versions of other kinases. Moreover, by applying the lysine photocaging to each kinase in a pathway, precise quantitative insights into the kinetics of kinase networks, and the substrates of individual kinases will be possible. Such quantitative insights in specifically and rapidly activated single-cell sub-networks will shed further light on the molecular pathways that lead to cell-to-cell variability and robustness and adaptation, and should allow us to rapidly constrain the experimental parameters in quantitative models of signal transduction (Aldridge et al., 2006; Asthagiri and Lauffenburger, 2001; Barkai and Leibler, 1997; Fujioka et al., 2006). It will also be possible to extend our approach to the wide range of other proteins that utilize NTP binding, allowing the temporal and spatial dependence of a wide range of biological processes to be manipulated and investigated.

Materials and methods

Reagents—The photo-caged lysine 1 was prepared as previously described (Gautier et al., 2010). TPA (12-O-tetradecanoylphorbol-13-acetate) was purchased from Cell Signaling. MEK inhibitor U0126 was purchased from Promega. Recombinant human epidermal growth factor (EGF) was purchased from Gibco. Western Blots were performed using antibodies against HA-tag (Sigma), Flag-tag (Cell Signaling), p44/42 MAPK (ERK1/2) (Cell Signaling), phospho-p44/42 MAPK (ERK1/2) (T202/Y204) (Cell Signaling), phospho-Elk1 (S383) (Cell signaling), phospho-p90RSK (S380) (Cell Signaling).

DNA constructs—The plasmids p4CMVE-U6-PylT (allowing the expression of the pyrrolysyl tRNA_{CUA} in mammalian cells) and pPCKRS-mCherry-TAG-EGFP-HA were described previously (Gautier et al., 2010). The plasmid pCR2.1/htRNA^{Tyr}_{CUA} for expressing the human tyrosine amber suppressor tRNA^{Tyr}_{CUA} in mammalian cell was a kind gift from T. Ashton Cropp (University of Maryland). The gene encoding MEK1 mutant fused to HA tag (MEK1-HA) was ligated into NheI and BssHII sites in the previously reported pPCKRS-p53-EGFP-HA plasmid (Gautier et al., 2010), allowing the simultaneous expression of MEK1-HA and the photo-caged lysyl-tRNA synthetase PCKRS. Plasmids for expressing the different MEK1 mutants were obtained by PCR mutagenesis and sequences were verified by DNA sequencing. A-MEK1-ΔN contains deletion Δ30-49; C-MEK1-ΔN contains deletion Δ30-49 and mutation K97TAG; D-MEK1-ΔN contains deletion Δ30-49 and mutation K97M; A-MEK1-DD contains mutations S218D and S222D; C-MEK1-DD contains mutations S218D, S222D and K97TAG; D-MEK1-ΔN contains mutations S218D, S222D and K97M; MEK1-K97M-HA contains mutation K97M. ERK2 gene was ligated into PstI and KpnI sites downstream of the enhanced green fluorescent protein (EGFP) gene in pEGFP-C (Clontech). Plasmids for expressing ERK2 mutants were obtained by PCR mutagenesis and sequences were verified by DNA sequencing. ERK2-AAA contains mutations T185A, E186A and Y187A. ERK2-M contains the deletion Δ174-177.

Cell culture and transfection—Human embryonic kidney 293ET cells were grown at 37° C. in 5% CO₂ atmosphere in DMEM+GlutaMAX-1 medium (Gibco) supplemented with 10% fetal bovine serum (FBS) and 1× pen-strep solution for 24 hours before transfection. Cells were transiently transfected with Genejuice (Novagen) according to the manufacturer's protocol. Cells were serum-starved (DMEM with 0.1% FBS) and grown with 2 mM of the photocaged lysine 1 for 24 h before analysis. Growth medium was replaced with fresh 1-free DMEM supplemented with 0.1% FBS before photo-activation experiments.

Photo-activation—Cells grown in 24-well plates were illuminated with a high power LED source module at 365 nm (Black-led-365, Prizmatix) placed underneath the plate, and then harvested for immunoblotting analysis.

Immunoblotting—Cells were washed with ice-cold phosphate buffer saline (PBS), then lysed with ice-cold universal lysis buffer (Roche) supplemented with protease inhibitors cocktail (Roche), 1 mM sodium vanadate, 5 mM sodium fluoride and 10 mM EDTA. Samples were resolved by SDS-PAGE and analyzed by immunoblotting with appropriate antibodies after transferring to nitrocellulose membranes.

Live cell imaging—Live cells grown in μ-Dish (Ibidi) were imaged at room temperature with an inverted Zeiss LSM 710 Laser Scanning Microscope equipped with a Plan Apochromat 63×/1.4 oil immersion objective. Photo-activation was performed for approximately 2 s (power: 1 mW/cm²) with an EXFO X-Cite 120 XL System employing a 120 W metal halide lamp with a UV filter (filter setting—excitation G 365, beam splitter FT 395, emission BP 445/50). EGFP was excited with a 488 nm argon laser, and emission was collected between 500-560 nm. The mean nuclear (Fn) and cytoplasmic (Fc) fluorescence intensities were quantified using ImageJ software to enable the F(n/c) ratio to be determined according to the formula: F(n/c)=(Fn−Fb)/(Fc−Fb), where Fb is the mean background fluorescence intensity.

### Example 7A

### Light-Activated Kinases Enable the Temporal Dissection of Signalling Networks in Living Cells

List of Supplementary Movies

Movie S1. Unveiling of ERK2 Nucleocytoplasmic Shuttling.

HEK293 cells cotransfected with plasmids encoding PCKRS, pyrrolysine tRNACUA, EGFP-ERK2 and C-MEK1-DD were grown in medium supplemented with 2 mM 1 and 0.1% FBS. Cells were illuminated using 365 nm light (2 s, 1 mW/cm2) at time t=0 min and U0126 (10 μM) was added after 8 min. On the right is shown the EGFP fluorescence of a representative cell. On the left is shown as a control the EGFP fluorescence of a representative cell non-treated with U0126. The graph shows normalized F(n/c) vs. time (mean of 10 representative experiments) with addition of U0126 after 8 min (black) and without addition of U0126 (grey).

Movie S2. Kinetics of Early EGFP-ERK2 Nuclear Translocation Upon Photoactivation of the Caged MEK1.

HEK293 cells co-transfected with plasmids encoding PCKRS, pyrrolysine tRNACUA, EGFP-ERK2 and C-MEK1-DD were grown in medium supplemented with 2 mM 1 and 0.1% FBS. Cells were illuminated using 365 nm light (2 s, 1 mW/cm2) at time t=0 s. The EGFP fluorescence of a representative cell is followed. Scale bar represents 10 μm. The graph on the right shows normalized F(n/c) vs. time (mean of 10 representative experiments).

We refer to FIG. 21, FIG. 22 and FIG. 23.

### REFERENCES TO EXAMPLE 7

Aldridge, B. B., Burke, J. M., Lauffenburger, D. A., and Sorger, P. K. (2006). Physicochemical modelling of cell signalling pathways. Nat Cell Biol 8, 1195-1203.
Ando, R., Mizuno, H., and Miyawaki, A. (2004). Regulated fast nucleocytoplasmic shuttling observed by reversible protein highlighting. Science 306, 1370-1373.
Asthagiri, A. R., and Lauffenburger, D. A. (2001). A computational study of feedback effects on signal dynamics in a mitogen-activated protein kinase (MAPK) pathway model. Biotechnol Prog 17, 227-239.
Banaszynski, L A., Chen, L C., Maynard-Smith, L A., Ooi, A. G., and Wandless, T. J. (2006). A rapid, reversible, and tunable method to regulate protein function in living cells using synthetic small molecules. Cell 126, 995-1004.
Barkai, N., and Leibler, S. (1997). Robustness in simple biochemical networks. Nature 387, 913-917.
Bashor, C. J., Heiman, N.C., Yan, S., and Lim, W. A. (2008). Using engineered scaffold interactions to reshape MAP kinase pathway signaling dynamics. Science 319, 1539-1543.
Bishop, A. C., Ubersax, J. A., Petsch, D. T., Matheos, D. P., Gray, N. S., Biethrow, J., Shimizu, E., Tsien, J. Z., Schultz, P. G., Rose, M. D., et al. (2000). A chemical switch for inhibitor-sensitive alleles of any protein kinase. Nature 407, 395-401.
Breitkreutz, A., Choi, H., Sharom, J. R., Boucher, L., Neduva, V., Larsen, B., Lin, Z. Y., Breitkreutz, B. J., Stark, C., Liu, G., et al. A global protein kinase and phosphatase interaction network in yeast. Science 328, 1043-1046.
Brunet, A., Roux, D., Lenormand, P., Dowd, S., Keyse, S., and Pouyssegur, J. (1999). Nuclear translocation of p42/p44 mitogen-activated protein kinase is required for growth factor-induced gene expression and cell cycle entry. EMBO J. 18, 664-674.
Burack, W. R., and Sturgill, T. W. (1997). The activating dual phosphorylation of MAPK by MEK is nonprocessive. Biochemistry 36, 5929-5933.
Burkard, M. E., Randall, C. L, Larochelie, S., Zhang, C., Shokat, K. M., Fisher, R. P., and Jallepalli, P. V. (2007). Chemical genetics reveals the requirement for Polo-like kinase 1 activity in positioning RhoA and triggering cytokinesis in human cells. Proc Natl Acad Sci 104, 4383-4388.
Chen, R. H., Sarnecki, C., and Bienis, J. (1992). Nuclear localization and regulation of erk- and rsk-encoded protein kinases. Mol Cell Biol 12, 915-927.
Choi, D. S., Wei, W., Deitchman, J. K., Kharazia, V. N., Lesscher, H. M., McMahon, T., Wang, D., Qi, Z. H., Sieghart, W., Zhang, C., et al. (2008). Protein kinase Cdelta regulates ethanol intoxication and enhancement of GABA-stimulated tonic current. J Neurosci 28, 11890-11899.
Cohen-Saidon, C., Cohen, A. A., Sigal, A., Liron, Y., and Alan, U. (2009). Dynamics and variability of ERK2 response to EGF in individual living cells. Mol Cell 36, 885-*893.
Costa; M., Marchi, M., Cardarelli, F., Roy, A., Beitram, F., Maffei, L., and Ratto, G. M. (2006). Dynamic regulation of ERK2 nuclear translocation and mobility in living cells. J Cell Sci 119, 4952-4963.
Cowley, S., Paterson, H., Kemp, P., and Marshall, C. J. (1994). Activation of MAP kinase kinase is necessary and sufficient for PC12 differentiation and for transformation of NIH 3T3 cells. Cell 77, 841-852.
Deiters, A. Principles and applications of the photochemical control of cellular processes. Chembiochem 11, 47-53.
Deiters, A. (2009). Light activation as a method of regulating and studying gene expression. Curr Opin Chem Biol 13, 678-686.
Dikic, I., Schlessinger, J., and Lax, I. (1994). PC12 cells overexpressing the insulin receptor undergo insulin-dependent neuronal differentiation. Curr Biol 4, 702-708.
Endo, M., Nakayama, K., Kaida, Y., and Majima, T. (2004). Design and synthesis of photochemically controllable caspase-3. Angew Chem Int Ed 43, 5643-5645.
Ferrell, J. E., Jr., and Shalt, R. R. (1997). Mechanistic studies of the dual phosphorylation of mitogen-activated protein kinase. J Biol Chem 272, 19008-19016.
Fujioka, A., Terai, K., Itoh, R. E., Aoki, K., Nakamura, T., Kuroda, S., Nishida, E., and Matsuda, M. (2006). Dynamics of the Ras/ERK MAPK cascade as monitored by fluorescent probes. J Biol Chem 281, 8917-8926.
Fukuda, M., Gotoh, I., Gotoh, Y., and Nishida, E. (1996). Cytoplasmic localization of mitogen-activated protein kinase kinase directed by its NH2-terminal, leucine-rich short amino acid sequence, which acts as a nuclear export signal. J Biol Chem 271, 20024-20028.
Fukuda, M., Gotoh, Y., and Nishida, E. (1997). Interaction of MAP kinase with MAP kinase kinase: its possible role in the control of nucleocytoplasmic transport of MAP kinase. EMBO J. 16, 1901-1908.
Gautier, A., Nguyen, D. P., Lusic, H., An, W., Deiters, A., and Chin, J. W. (2010). Genetically encoded photocontrol of protein localization in mammalian cells. J Am Chem Soc 132, 4086-4088.
Ghosh, M., Song, X., Mouneimne, G., Sidani, M., Lawrence, D. S., and Condeelis, J. S. (2004). Cofilin promotes actin polymerization and defines the direction of cell motility. Science 304, 743-746.
Hahn, M. E., and Muir, T. W. (2004). Photocontrol of Smad2, a multiphosphorylated cell-signaling protein, through caging of activating phosphoserines. Angew Chem Int Ed 43, 5800-5803.
Huang, S., Jiang, Y., Li, Z., Nishida, E., Mathias, P., Lin, S., Ulevitch, R. J., Nemerow, G. R., and Han, J. (1997). Apoptosis signaling pathway in T cells is composed of ICE/Ced-3 family proteases and MAP kinase kinase 6b. Immunity 6, 739-749.
Justman, Q. A., Serber, Z., Ferrell, J. E., Jr., El-Samad, H., and Shokat, K. M. (2009). Tuning the activation threshold of a kinase network by nested feedback loops. Science 324, 509-512.
Karginov, A. V., Ding, F., Kota, P., Dokholyan, N. V., and Hahn, K. M. (2010). Engineered allosteric activation of kinases In living cells. Nat Biotechnol 28, 743-747.
Khokhlatchev, A. V., Canagarajah, B., Wilsbacher, J., Robinson, M., Atkinson, M., Goldsmith, E., and Cobb, M. H. (1998). Phosphorylation of the MAP kinase ERK2 promotes its homodimerization and nuclear translocation. Cell 93, 605-615.
Kim, J. S., Lilley, B. N., Zhang, C., Shokat, K. M., Sanes, J. R., and Zhen, M. (2008). A chemical-genetic strategy reveals distinct temporal requirements for SAD-1 kinase in neuronal polarization and synapse formation. Neural Dev 3, 23.
Kim, K., Nose, K., and Shibanuma, M. (2000). Significance of nuclear relocalization of ERK1/2 in reactivation of c-fos transcription and DNA synthesis in senescent fibroblasts. J Biol Chem 275, 20685-20692.
Larochelle, S., Batliner, J., Gamble, M. J., Barboza, N. M., Kraybill, B. C., Blethrow, J. D., Shokat, K. M., and Fisher, R. P. (2006). Dichotomous but stringent substrate selection by the dual-function Cdk7 complex revealed by chemical genetics. Nat Struct Mol Blol 13, 55-62.
Lawrence, D. S. (2005). The preparation and in vivo applications of caged peptides and proteins. Curr Opin Chem Biol 9, 570-575.
Lee, H. M., Larson, D. R., and Lawrence, D. S. (2009). Illuminating the chemistry of life: design, synthesis, and applications of “caged” and related photoresponsive compounds. ACS Chem Biol 4, 409-427.
Lemke, E. A., Summerer, D., Geierstanger, B. H., Brittain, S. M., and Schultz, P. G. (2007). Control of protein phosphorylation with a genetically encoded photocaged amino acid. Nat Chem Biol 3, 769-772.
Lenormand, P., Sardet, C., Pages, G., L'Allemain, G., Brunet, A., and Pouyssegur, J. (1993). Growth factors induce nuclear translocation of MAP kinases (p42mapk and p44mapk) but not of their activator MAP kinase kinase (p45mapkk) in fibroblasts. J Cell Biol 122, 1079-1088.
Levskaya, A., Weiner, O. D., Lim, W. A., and Voigt, C. A. (2009). Spatiotemporal control of cell signalling using a light-switchable protein interaction. Nature 461, 997-1001.
Li, S., Makovets, S., Matsuguchl, T., Blethrow, J. D., Shokat, K. M., and Blackburn, E. H. (2009). Cdk1-dependent phosphorylation of Cdc13 coordinates telomere elongation during cell-cycle progression. Cell 136, 50-61.
Lidke, D. S., Huang, F., Post, J. N., Rieger, B., Wilsbacher, J., Thomas, J. L, Pouyssegur, J., Jovin, T. M., and Lenormand, P. (2010). ERK nuclear translocation is dimerization-independent but controlled by the rate of phosphorylation. J. Biol Chem 285, 3092-3102.
Malleshalah, M. K., Shahrezaei, V., Swain, P. S., and Michnick, S. W. The scaffold protein Step 5 directly controls a switch-like mating decision in yeast. Nature 465, 101-105.
Manning, G., Whyte, D. B., Martinez, R., Hunter, T., and Sudarsanam, S. (2002). The protein kinase complement of the human genome. Science 298, 1912-1934.
Mansour, S. J., Molten, W. T., Hermann, A. S., Candia, J. M., Rong, S., Fukasawa, K., Vande Woude, G. F., and Ahn, N. G. (1994). Transformation of mammalian cells by constitutively active MAP kinase kinase. Science 265, 966-970.
Markevich, N. I., Hoek, J. B., and Kholodenko, B. N. (2004). Signaling switches and bistability arising from multisite phosphorylation in protein kinase cascades. J Cell Biol 164, 353-359.
Minden, A., Lin, A., McMahon, M., Lange-Carter, C., Derijard, B., Davis, R. J., Johnson, G. L, and Karin, M. (1994). Differential activation of ERK and JNK mitogen-activated protein kinases by Raf-1 and MEKK. Science 266, 1719-1723.
Payne, D. M., Rossomando, A. J., Martino, P., Erickson, A. K., Her, J. H., Shabanowitz, J., Hunt, D. F., Weber, M. J., and Sturgill, T. W. (1991). Identification of the regulatory phosphorylation sites in pp 42/mitogen-activated protein kinase (MAP kinase). EMBO J. 10, 885-892.
Pellois, J. P., Hahn, M. E., and Muir, T. W. (2004). Simultaneous triggering of protein activity, and fluorescence. J Am Chem Soc 126, 7170-7171.
Pellois, J. P., and Muir, T. W. (2005). A Ligation and photorelease strategy for the temporal and spatial control of protein function in living cells. Angew Chem int Ed 44, 5713-5717.
Qiao, Y., Molina, H., Pandey, A., Zhang, J., and Cole, P. A. (2006). Chemical rescue of a mutant enzyme in living cells. Science 311, 1293-1297.
Raingeaud, J., Gupta, S., Rogers, J. S., Dickens, M., Han, J., Ulevitch, R. J., and Davis, R. J. (1995). Pro-inflammatory cytokines and environmental stress cause p38 mitogen-activated protein kinase activation by dual phosphorylation on tyrosine and threonine. J Biol Chem 270, 7420-7426.
Rubinfeld, H., Hanoch, T., and Seger, R. (1999). Identification of a cytoplasmic-retention sequence in ERK2. J Biol Chem 274, 30349-30352.
Salazar, C., and Hofer, T. (2009). Multisite protein phosphorylation—from molecular mechanisms to kinetic models. FEBS J 276, 3177-3198.
Shaul, Y. D., and Seger, R. (2007). The MEK/ERK cascade: from signaling specificity to diverse functions. Biochim Biophys Acta 1773, 1213-1226.
Spencer, D. M., Wandless, T. J., Schreiber, S. L, and Crabtree, G. R. (1993). Controlling signal transduction with synthetic ligands. Science 262, 1019-1024.
Traverse, S., Seedorf, K., Paterson, H., Marshall, C. J., Cohen, P., and Ullrich, A. (1994). EGF triggers neuronal differentiation of PC12 cells that overexpress the EGF receptor. Curr Biol 4, 694-701.
Ventura, J. J., Hubner, A., Zhang, C., Flavell, R. A., Shokat, K. M., and Davis, R. J. (2006). Chemical genetic analysis of the time course of signal transduction by JNK. Mol Cell 21, 701-710.
Volmat, V., Camps, M., Arkinstall, S., Pouyssegur, J., and Lenormand, P. (2001). The nucleus, a site for signal termination by sequestration and inactivation of p42/p44 MAP kinases. J Cell Sci 114, 3433-3443.

Primer list
mGFPHindamf
5′-GCTCAAGCTTCACCATGGCACTAGCAATTAGCCATGGTGAGCAAG
GGCGAGGAGCTGTTCACCG-3′
AG27
5′-TCCGGTGGATCCTTATCATTAAGCGTAATCTGGAACATCGTATGGGT
ACATCTTGTACAGCTCGTCCATGC-3′
3367bkf
5′-GAAGGTACCCGATCGCCGCGGACCGGTTTAATTAAGCGGCCGCTA
GTTATTAATAGTAATCAATTACGGGGTCATTAGTTCATAGCC-3′
3367bkr
5′-ATAGCGGCCGCTTAATTAAACCGGTCCGCGGCGATCGGGTACCAT
GCATGGCGGTAATACGGTTATCCACAGAATCAGGGGATAACGCAGGA
AAGAAC-3′
KpnpvukSf
5′-GAAGGTACCCGATCGACTAGTTATTAATAGTAATCAATTACGGGGTC
ATTAG-3′
AgesacKSr
5′-GAAACCGGTCCGCGGAAGCCATAGAGCCCACCGCATCCCCAGC
ATG-3′
AG40
5′-TAAACTTAAGCTTGCCACCATGGACTACAAGGACGAC-3′
AG43
5′-GGCACAGGTTCTTGTCCACCCGGAAAATCTGCTTGGACAGCTCGGTG
TCGTTGTTGATGCCGAACCGCTCCACGTACTC-3′
AG42
5′-GGTGGACAAGAACCTGTGCCTGCGGCCTATGCTGAGCCCCACCCT
GTGCAACTACATGCGGAAACTGGACAGAATC-3′
AG41
5′-ATCTGCAGAATTCCACCACACTGGACTAGTGGATCCTTATC-3′
AG16
5′-ATGCTAGGATCCTTAATTAAACTAGTCTAGTTATTAATAGTAATCAA
TTACGG-3′
AG17
5′-ATGCTAAGATCTGTCCCGTTGATTTTGGTGCC-3′
AG30
5′-ATGCTAGGATCCAGATCTTCTAGACTCGAAGGAAACCTG-3′
AG20
5′-ATGCTACAATTGCCGCGGGAATTCGCTAGCAAAAACCGCACTTGTC
CGGAAACC-3′
AG52
5′-CAGATCCGCTAGCACCGGTGCGATCGCACCATGGAGGAGCCGCA
GTCAGATCCTAG-3′
AG53
5′-GCTCGAGATCTGAGTCCGGATGGCGCGCCGTCTGAGTCAGGCCCTT
CTG-3′
AG54
5′-GGCGCGCCATCCGGACTCAGATCTCGAGCTCAAGC-3′
AG55
5′-TAAACAAGTTAACAACAACAATTGCATTC-3′
AG58
5′-GTTGTTGGGCAGTGCTCGGGCAGTGCTCCCTGGGGGCAGCTCGTG
GTG-3′
AG56
5′-CGAGCACTGCCCAACAACACCAG-3′
AG57
5′-GTTGTTGGGCAGTGCTCGCTAAGTGCTCCCTGGG-3′
AG95
5′-AGATCCGCTAGCACCGGTGCGATCGCACCATGGCTAGCATGACTG
GTGGACAG-3′
AG96
5′-CGGATGGCGCGCCTTATCATTAAGCGTAATCTGGAACATCGTATGGG
TACATCTCGAGGCAGCCGGATCCTTTG-3′
AG97
5′-TTGATCCAGTTTCTTTTTCTACGCCTGGCCC-3′
AG98
5′-TTGATCCAGTTTCTTTTTGGCCGCCTGGCCC-3′
AG99
5′-AAAAAGAAACTGGATCAAG-3′

### REFERENCES

1) (a) Deiters, A. Chembiochem 2010, 11, 47-53; (b) Lee, H. M.; Larson, D. R.; Lawrence, D. S. ACS Chem. Biol. 2009, 4, 409-427; (c) Deiters, A. Curr. Opin. Chem. Biol. 2009, 13, 678-686; (d) Young, D. D.; Deiters, A. Org. Biomol. Chem. 2007, 5, 999-1005.
(2) Mayer, G.; Heckel, A. Angew. Chem. Int. Ed. 2006, 45, 4900-4921.
(3) Endo, M.; Nakayama, K.; Kaida, Y.; Majima, T. Angew. Chem. Int. Ed. 2004, 43, 5643-5645.
(4) Pellois, J. P.; Hahn, M. E.; Muir, T. W. J. Am. Chem. Soc. 2004, 126, 7170-7171.
(5) Hahn, M. E.; Muir, T. W. Angew. Chem. Int. Ed. 2004, 43, 5800-5803.
(6) Pellois, J. P.; Muir, T. W. Angew. Chem. Int. Ed. 2005, 44, 5713-5717.
(7) (a) Wu, N.; Deiters, A.; Cropp, T. A.; King, D.; Schultz, P. G. J. Am. Chem. Soc. 2004, 126, 14306-14307; (b) Deiters, A.; Groff, D.; Ryu, Y. H.; Xie, J. M.; Schultz, P. G. Angew. Chem. Int. Ed. 2006, 45, 2728-2731; (c) Lemke, E. A.; Summerer, D.; Geierstanger, B. H.; Brittain, S. M.; Schultz, P. G. Nat. Chem. Biol. 2007, 3, 769-772.
(8) Chen, P. R.; Groff, D.; Guo, J. T.; Ou, W. J.; Cellitti, S.; Geierstanger, B. H.; Schultz, P. G. Angew. Chem. Int. Ed. 2009, 48, 4052-4055.
(9) Stewart, M. Nat. Rev. Mol. Cell. Biol. 2007, 8, 195-208.
(10) Yang, X. J. Oncogene 2005, 24, 1653-1662.
(11) Neumann, H.; Peak-Chew, S. Y.; Chin, J. W. Nat. Chem. Biol. 2008, 4, 232-234.
(12) Chin, J. W.; Martin, A. B.; King, D. S.; Wang, L.; Schultz, P. G. Proc. Natl. Acad. Sci. 2002, 99, 11020-11024.
(13) Mukai, T.; Kobayashi, T.; Hino, N.; Yanagisawa, T.; Sakamoto, K.; Yokoyama, S. Biochem. Biophys. Res. Commun. 2008, 371, 818-822.
(14) Galli, G.; Hofstetter, H.; Birnstiel, M. L. Nature 1981, 294, 626-631.
(15) Xia, X. G.; Zhou, H. X.; Ding, H. L.; Affar, E. B.; Shi, Y.; Xu, Z. S, Nucl. Acids Res. 2003, 31, e100.
(16) Nguyen, D. P.; Lusic, H.; Neumann, H.; Kapadnis, P. B.; Deiters, A.; Chin, J. W. J. Am. Chem. Soc. 2009, 131, 8720-8721.
(17) Robbins, J.; Dilworth, S. M.; Laskey, R. A.; Dingwall, C. Cell 1991, 64, 615-623.
(18) (a) Liang, S. H.; Clarke, M. F. J. Biol. Chem. 1999, 274, 32699-32703; (b) O'keefe, K.; Li, H. P.; Zhang, Y. P. Mol. Cell. Biol. 2003, 23, 6396-6405.
(19) Lusic, H.; Deiters, A. Synthesis 2006, 2147-2150.
(20) Neumann, H.; Hancock, S. M.; Buning, R.; Routh, A.; Chapman, L.; Somers, J.; Owen-Hughes, T.; van Noort, J.; Rhodes, D.; Chin, J. W. Molecular Cell 2009, 36, 153-163.
(21) Rackham, O.; Chin, J. W. Nat. Chem. Biol. 2005, 1, 159-166.

TABLE I
Sequences of PylRS variants selected for the specific incorporation
of the caged lysine according to the invention.

| | Mutations | | | | |
|---|---|---|---|---|---|
| Clones | 241 | 267 | 271 | 274 | 313 |
| PylRS | M | A | Y | L | C |
| 1 | Y | G | C | M | C |
| 2 | M | A | Y | V | V |
| 3 | F | A | A | I | A |
| 4 | M | A | T | V | C |
| 5 | M | S | I | V | C |
| 6 | F | S | C | M | A |
| 7 | F | S | C | M | C |
| 8 | F | S | T | M | C |
| 9 | F | A | T | I | A |
| 10 | F | S | T | M | C |
| 11 | M | A | A | V | C |
| 12 | M | A | A | V | A |
| 13 | M | T | I | M | S |
| 14 | M | G | A | M | A |
| 15 | F | A | C | I | A |
| 16 | M | A | I | M | A |
| 17 | M | S | A | A | A |

What is claimed:

1. A caged lysine, wherein the caged lysine is according to Formula (I) 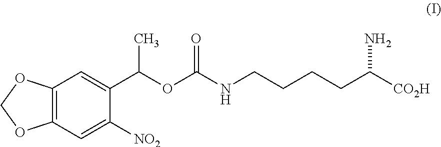 [SMILES: CC(OC(=O)NCCCC[C@H](N)C(=O)O)c1cc2c(cc1[N+](=O)[O-])OCO2]
or salts thereof.

2. A polypeptide comprising a caged lysine according to claim 1.

3. A polypeptide according to claim 2 wherein said caged lysine is present at a position in the polypeptide corresponding to a lysine residue in the wild type polypeptide.

4. A polypeptide according to claim 2 which is a nucleotide triphosphate binding protein.

5. A polypeptide according to claim 4 which is a kinase.

6. A polypeptide according to claim 5 wherein the caged lysine is present in the catalytic site of said kinase.

7. A polypeptide according to claim 6 wherein decaging of the lysine permits kinase activity of said polypeptide.

8. A method of making a polypeptide comprising a caged lysine according to claim 1, said method comprising arranging for the translation of a RNA encoding said polypeptide, wherein said RNA comprises an orthogonal codon, wherein said translation is carried out in the presence of tRNA recognising said orthogonal codon and capable of being charged with caged lysine according to claim 1, and in the presence of a tRNA synthetase capable of charging said tRNA with caged lysine according to claim 1, and in the presence of caged lysine according to claim 1.

9. A method according to claim 8 wherein the tRNA synthetase comprises pyrollysyl-tRNA synthetase with mutations relative to the wild type sequence in one to five positions according to Table I wherein the mutation(s) are present at positions corresponding to one to five residues selected from M241, A267, Y271, L274 and C313.

10. A method according to claim 9 wherein the tRNA synthetase comprises four mutations, wherein the mutations are M241F, A267S, Y271C and L274M.

11. A method according to claim 8 wherein the orthogonal codon is an amber codon (TAG).

12. A method according to claim 11 wherein the orthogonal tRNA is PyltRNAcuA.

13. (canceled)

14. (canceled)

15. (canceled)

16. A pyrollysyl-tRNA synthetase with mutations relative to the wild type sequence in one to five positions according to Table I wherein the mutation (s) are present at positions corresponding to one to five residues selected from M241, A267, Y271, L274 and C313.

17. The orthogonal pyrollysyl-tRNA synthetase according to claim 16, comprising four mutations, wherein the mutations are M241F, A267S, Y271C and L274M.

18. (canceled)
